(19) United States
(12) Patent Application Publication
(10) Pub. No.: US 2024/0400974 A1
(43) Pub. Date: Dec. 5, 2024

(54) NOVEL YEAST STRAINS

(71) Applicant: BISY GMBH, Hofstaetten an der Raab (AT)

(72) Inventors: Anton GLIEDER, Hofstaetten an der Raab (AT); Claudia RINNOFNER, Hart bei Graz (AT); Stefan ERTL, Hofstaetten an der Raab (AT); Katharina EBNER, Hofstaetten an der Raab (AT); Moritz BÜRGLER, Graz (AT); Astrid RADKOHL, Hofstaetten an der Raab (AT)

(73) Assignee: BISY GMBH, Hofstaetten an der Raab (AT)

(21) Appl. No.: 18/270,070

(22) PCT Filed: Dec. 29, 2021
(86) PCT No.: PCT/EP2021/087763

§ 371 Date: Jun. 28, 2023

(30) Foreign Application Priority Data
Dec. 29, 2020 (EP) 20217516.2

(51) Int. Cl. C12N 1/16; C12N 15/81; C12P 21/00; C12R 1/645

(52) U.S. Cl. CPC C12N 1/16; C12N 15/815; C12P 21/00; C12R 2001/645

(57) ABSTRACT

A genetically modified Komagataella phaffii yeast cell for expression of a Protein or Polypeptide of Interest (POI). comprising in its genome a recombinant nucleic acid sequence encoding a POI, and a genetic modification in the open reading frame at any one or more of position 949930 on chromosome 1 (genbank LT962476.1), position 65654 on chromosome 2 (genbank LT962477.1), position 1303485 on chromosome 4 (genbank LT962479.1), position 1323758 on chromosome 1 (genbank LT962476.1) and/or position 1491140 on chromosome 4 (genbank LT962479.1).

This application is a National Phase Application under 35 U.S.C § 371 of International Application PCT/EP2021/087763, filed Dec. 29, 2021, the entirety of which is incorporated herein by reference. International Application PCT/EP2021/087763 claims the benefit of European Patent Application No. 20217516.2, filed Dec. 29, 2020.

Pursuant to 37 C.F.R. 1.821 (c), a sequence listing is submitted herewith as an ASCII compliant text file named “REDL.P0008US-Sequence Listing.txt”, created on Jun. 19, 2023, and having a size of ˜97 kilobytes. The content of the aforementioned file is hereby incorporated by reference in its entirety.

### FIELD OF THE INVENTION

The present invention relates to genetically modified yeast cells, in particular Komagataella phaffii yeast cells, that are useful for the production of a protein or polypeptide of interest (POI). Specifically, the invention relates to the genetically modified yeast strains and their use in production of a variety of POIs.

### BACKGROUND ART

Komagataella phaffii (syn. Pichia pastoris ) is a versatile and cost-effective microbial protein production system with beneficial characteristics such as strong gene expression, effective secretion and rapid biomass growth, which result in high titers and productivities of both intracellular and secreted recombinant proteins (Cregg et al., 2000). The feasibility of efficient (multi-) gene expression and robust growth on cheap and simple media is also beneficial (Lin-cereghino et al., 2008; Lin-Cereghino and Lin-Cereghino, 2007). A variety of P. pastoris platform technologies has been developed so far (Vogl et al., 2018b; Weninger et al., 2016). Integration of genes into the genome of P. pastoris is based on either homologous recombination or non-homologous end-joining (NHEJ, (Näätsaari et al., 2012); Weninger et al., 2018). Multicopy integration is commonly used to increase titres of recombinant bio-molecules. This can either be achieved by using plasmids with several copies or by screening of many clones for spontaneously higher copy numbers. Depending on the design of the flanking regions (i.e. length, type, structure), untargeted (random) genome integration mediated by NHEJ or locus-specific integration becomes prevalent. While random integration can be a useful tool to prevent multiple integration events at single genomic loci (leading to instability), expression levels of randomly integrated genes may be influenced not only by copy number but also by the integration locus. Random integration can influence the expression levels of endogenous genes due to knock out or gene silencing events and lead to unexpectedly high or low production levels. However, it is still unknown if single specific integration sites or various different sites in the genome can cause such effects and there are no reports so far where new and more efficient platform strains were developed for other alternative targets.

Such hyper-producing or super-producing exceptional transformants were reported by (Brooks et al., 2013), who showed that a small number of highly expressing “Jackpot” clones can be isolated from a large number of clones in screening. The potential of ectopic integrations has also been demonstrated by (Larsen et al., 2013), who used a restriction enzyme mediated insertion strategy to identify gene products involved in the secretion process of P. pastoris . 12 genes were identified to increase the secretion efficiency of a β-galactosidase reporter. However, the best four bgs mutants were found to differ in their ability to enhance reporter protein secretion and for some mutants perhaps the uptake of substrate into the cell was facilitated and therefore enhanced signals in colorimetric β-galactosidase assays, rather than efficient secretion of the β-galactosidase reporter. Nevertheless, one mutant, bgs13, showed to affect the secretion of a wider range of recombinant proteins, suggesting the gene to play a more general role in protein export. More recently, Bgs13p was suggested to facilitate regulation of unfolded protein response and protein sorting on a global scale (Naranjo et al., 2019). Also, Vogl et al., (2018a) showed a few enhanced producing clones (<10%) with ectopically integrated cassettes, spanning a 25-fold range in expression and surpassing specifically integrated reference strains up to 6-fold. No details about those clones or any possible advantages for alternative targets were reported. Based on the findings in that study and previously published literature it was concluded, that Jackpot clones for different specific targets (POIs) can be identified by extensive screening. However, copy number variation, genomic integration sites, and even genomic deletions and rearrangements altogether have unique effects for different proteins of interest (Vogl et al., 2018a). Similarly, in a master thesis project at TU Graz (published poster from master thesis of C. Winkler, working group Pichler, IMBT), interesting Jackpot clones were obtained using randomly integrated linear DNA fragments. The potential of Jackpot clones is also highlighted by (Gasser et al., 2014), claiming under-expression of the P. pastoris genes FLO8, HCH1 and SCJ1 to increase the yield of model proteins. Integration event induced changes in recombinant protein production in P. pastoris were also studied by (Schwarzhans et al., 2016) by whole genome sequencing. However, in this study the term jackpot strains refers to strains with a gene copy number >10, similar to the study of Aw and Polizzi (2013). In those studies (and similar to the study of Vogl et al 2018a), strains in the high producer group displayed a markedly higher GCN and expression level than the reference clone with a GCN and normalized GFP expression of one. Similarly (Sunga et al., 2008) described so-called ‘jackpot’ clones with >10 copies of the expression vector to represent 5-6% of selected clones and to have a proportional increase in recombinant protein. In spite of the comprehensive NextGen genome sequencing efforts by Schwarzhans et al (2016) and Vogl et al (2018a), no reliable way to construct superproducer cells without extensive screening of transformants was found so far.

US2019/0390228A1 discloses a modified Pichia pastoris strain comprising a deletion of the Sec72 gene, which improved protein secretion.

US2005/0170452A1 discloses deleting the alg9 gene to generate yeast cells producing modified N-gylcans. Deletion of the alg9 gene created a host cell which produces N-glycans with one or two additional mannoses, respectively, on the 1,6 arm.

Despite the demonstrated potential of Jackpot clones, the generation of production strains is still a time-consuming process, which requires many iterative and repetitive steps. There is no general solution to obtain high titers of recombinant proteins so far. Most efforts to construct efficient industrial production clones still rely on original cell lines which were available for gene expression since the eighties (Cregg et al., 1985) and in spite of a few reported expression enhancing gene disruptions, such strains surprisingly were not repeatedly reported to be useful platform strains in following expression strain construction efforts. There is a lack of highly efficient next generation host strains. But no systematic and widely applicable way to generate such super-producing strains was found so far. A generic biology/bioinformatics approach for genetic analysis of spontaneously occurring super-producing strains has not yet been demonstrated and genetic mechanisms underlying especially good expressing strains mostly remain undiscovered; most probably due to limited availability of such clones and the complexity and mostly unknown mechanisms behind efficient protein secretion by eukaryotic hosts in general. Costs of bioinformatic analysis and high demands in big data interpretation thereof also caused bottle necks which are still not sufficiently resolved.

Thus, there is an unmet need in the art for a next generation of K. phaffii strains, which allow the efficient production of a plurality of recombinant proteins with little effort and high success rates and does not require extensive transformation combined with high throughput screening of state of the art P. pastoris strain transformants.

### SUMMARY OF INVENTION

It is an objective of the present invention to provide improved means of producing recombinant proteins in yeast strains. It is a specific objective of the present invention to provide improved yeast strains of the genus Komagataella, which allow production of recombinant proteins in high yields.

The objective is solved by the subject matter of the present invention.

According to the invention there is provided a genetically modified Komagataella phaffii yeast cell for expression of a Protein or Polypeptide of Interest (POI), comprising in its genome a recombinant nucleic acid sequence encoding a POI, and a genetic modification in the open reading frame at position 949930 on chromosome 1 (genbank LT962476.1), position 65654 on chromosome 2 (genbank LT962477.1), position 1303485 on chromosome 4 (genbank LT962479.1), position 1323758 on chromosome 1 (genbank LT962476.1) and/or position 1491140 on chromosome 4 (genbank LT962479.1).

Specifically, said genetic modification is an inactivating modification.

Specifically, the yeast cells provided herein comprise a genetic modification in any one or all of the open reading frames at position 949930 on chromosome 1 (genbank LT962476.1), position 65654 on chromosome 2 (genbank LT962477.1), position 1303485 on chromosome 4 (genbank LT962479.1), position 1323758 on chromosome 1 (genbank LT962476.1) and/or position 1491140 on chromosome 4 (genbank LT962479.1). Specifically, the yeast cells described herein comprise a genetic modification in at least 1, 2, 3, or 4 or all 5 of said reading frames.

In a specific embodiment, the genetically modified yeast cell described herein comprises a genetic modification around, e.g. within 1, 2, 3, 4, 5, 6, 7, 8, 9, 10, 15, 20, 25, 30, 35, 40, 45, 50, or 100, 200, 300, 400 or 500 nucleotides upstream or downstream, position 65654 on chromosome 2 (genbank LT962477.1), position 949930 on chromosome 1 (genbank LT962476.1), position 1303485 on chromosome 4 (genbank LT962479.1), position 1323758 on chromosome 1 (genbank LT962476.1) or position 1491140 on chromosome 4 (genbank LT962479.1).

In a further specific embodiment, the genetically modified yeast cell described herein comprises a genetic modification in the gene at any one or more or all of the positions selected from the group consisting of position 65654 on chromosome 2 (genbank LT962477.1), position 949930 on chromosome 1 (genbank LT962476.1), position 1303485 on chromosome 4 (genbank LT962479.1), position 1323758 on chromosome 1 (genbank LT962476.1) and position 1491140 on chromosome 4 (genbank LT962479.1).

In a further specific embodiment, the genetically modified yeast cell described herein comprises a genetic modification at any one or more or all of the positions selected from the group consisting of position 65654 on chromosome 2 (genbank LT962477.1), position 949930 on chromosome 1 (genbank LT962476.1), position 1303485 on chromosome 4 (genbank LT962479.1), position 1323758 on chromosome 1 (genbank LT962476.1) and position 1491140 on chromosome 4 (genbank LT962479.1).

Specifically, the yeast cell described herein has a genetic modification as described herein, specifically an inactivating mutation, in an endogenous gene selected from the group consisting of ALG9 (SEQ ID NO:14), SRB8 (SEQ ID NO:15), ACIB2EUKG772803 (SEQ ID NO:16), KAP123 (SEQ ID NO:17) and FLO400 (SEQ ID NO:18).

Specifically, the yeast cell described herein has a genetic modification as described herein, specifically an inactivating mutation, in an endogenous gene encoding a protein selected from the group consisting of ALG9 (SEQ ID NO:21), SRB8 (SEQ ID NO:20), ACIB2EUKG772803 (SEQ ID NO:22), KAP123 (SEQ ID NO:23) and FLO400 (SEQ ID NO:24). Specifically, said genetic modification prevents expression of functional ALG9 (SEQ ID NO:21), SRB8 (SEQ ID NO:20), ACIB2EUKG772803 (SEQ ID NO:22), KAP123 (SEQ ID NO:23) and/or FLO400 (SEQ ID NO:24).

Specifically, the genetic modification is a deletion and/or insertion of one or more bases, and/or a fusion of a chromosomal DNA sequence with a sequence of another chromosome.

In a specific embodiment, the genetic modification is a fusion of chromosomal sequences. Specifically, it is a fusion of at least two chromosomes. Specifically, it is a fusion of a DNA sequence at or around, e.g. within about 10, 50 or 100 bases, of any one of the positions described herein with a DNA sequence of a different chromosome.

According to a specific example, it is a fusion between chromosome 1 and chromosome 4 of K. phaffii . Specifically, a fusion of the chromosomal sequence at or around, e.g. within about 10, 50 or 100 bases, position 1323758 of chromosome 1 (genbank LT962476.1) and the chromosomal sequence at or around, e.g. within about 10, 50 or 100 bases, position 1491140 of chromosome 4 (genbank LT962479.1).

In a specific embodiment, the genetic modification is a knock-out, specifically of a part of the gene or of the whole gene.

In a specific embodiment, the genetic modification is at least one point mutation.

In a specific embodiment, the genetic modification is a modification, specifically an inactivating mutation, in the ALG9 gene, the SRB8 gene, and/or the ACIB2EUKG772803 gene. Specifically, the genetic modification is a modification preventing expression of a functional protein from the ALG9 gene, the SRB8 gene, and/or the ACIB2EUKG772803 gene.

In a specific embodiment, the genetic modification is caused by a genomic rearrangement within a chromosome or exchange of DNA sequences between different chromosomes.

In a further specific embodiment, the genetic modification is a deletion of 1 or more bases. Specifically, it is a deletion of 2, 3, 4, 5, 6, 7, 8, 9, 10, 11, 12, 13, 14, 15, 16, 17, 18, 19, 20, 25, 30, 35, 40, 45, 50, 60, 70, 80, 90, 100, 150, 200, 250, 300, 350, 400 or 500 nucleotides or more.

In yet a further specific embodiment, the deletion is a deletion of at least 50%, preferably at least 90%, of the gene at position 65654 on chromosome 2 (genbank LT962477.1), position 949930 on chromosome 1 (genbank LT962476.1), position 1303485 on chromosome 4 (genbank LT962479.1), position 1323758 on chromosome 1 (genbank LT962476.1) or position 1491140 on chromosome 4 (genbank LT962479.1).

In a specific embodiment, the genetic modification is an insertion or replacement of 1 or more bases. Specifically, it is an insertion or replacement of 2, 3, 4, 5, 6, 7, 8, 9, 10, 11, 12, 13, 14, 15, 16, 17, 18, 19, 20, 25, 30, 35, 40, 45, 50, 60, 70, 80, 90, 100, 150, 200, 250, 300, 350, 400 or 500 nucleotides or more.

In a further specific embodiment, the genetic modification is integration of the recombinant nucleic acid sequence encoding the POI.

In a specific embodiment, the sequence encoding the POI is comprised in an expression cassette, preferably comprising the following functional regions:
a. a promoter active in yeast of the genus Komagataella,
b. the nucleic acid sequence encoding the POI, operably linked to said promoter,
c. transcription termination sequences, and optionally
d. a selection marker, preferably an antibiotics resistance gene or carbon source utilization marker.

Further provided herein is a method of producing a recombinant Protein or Polypeptide of Interest (POI) comprising the steps of:
a. providing the genetically modified yeast cell described herein,
b. cultivating said genetically modified yeast cell in a culture medium under conditions that allow for expression of the POI, and
c. isolating the POI from the cells or the culture medium.

Further provided herein is a genetically modified Komagataella phaffi yeast cell for expression of a variety of Proteins or Polypeptides of Interest (POIs), comprising in its genome
a. a landing pad, comprising an empty expression cassette comprising target sequences for homologous recombination, and optionally any one or more of a selection marker or reporter protein, a stuffer fragment, a promoter 5′ of said stuffer fragment, and a transcription terminator 3′ of said stuffer fragment; and
b. a genetic modification in the open reading frame at position 949930 on chromosome 1 (genbank LT962476.1), position 65654 on chromosome 2 (genbank LT962477.1), position 1303485 on chromosome 4 (genbank LT962479.1), position 1323758 on chromosome 1 (genbank LT962476.1), and/or position 1491140 on chromosome 4 (genbank LT962479.1).

Specifically, the yeast cells comprising a landing pad provided herein for expression of a variety of POIs comprise a genetic modification in any one or all of the open reading frames, or genes, at position 949930 on chromosome 1 (genbank LT962476.1), position 65654 on chromosome 2 (genbank LT962477.1), position 1303485 on chromosome 4 (genbank LT962479.1), position 1323758 on chromosome 1 (genbank LT962476.1) and/or position 1491140 on chromosome 4 (genbank LT962479.1). Specifically, the yeast cells described herein comprise a genetic modification in at least 1, 2, 3, or 4 or all 5 of said reading frames.

In a specific embodiment, the yeast cells provided herein for expression of a variety of POIs comprises a genetic modification around, e.g. within 1, 2, 3, 4, 5, 6, 7, 8, 9, 10, 15, 20, 25, 30, 35, 40, 45 or 50 nucleotides upstream or downstream, position 65654 on chromosome 2 (genbank LT962477.1), position 949930 on chromosome 1 (genbank LT962476.1), position 1303485 on chromosome 4 (genbank LT962479.1), position 1323758 on chromosome 1 (genbank LT962476.1) or position 1491140 on chromosome 4 (genbank LT962479.1).

In a further specific embodiment, the yeast cells comprising a landing pad provided herein for expression of a variety of POIs comprises a genetic modification at position 65654 on chromosome 2 (genbank LT962477.1), position 949930 on chromosome 1 (genbank LT962476.1), position 1303485 on chromosome 4 (genbank LT962479.1), position 1323758 on chromosome 1 (genbank LT962476.1) or position 1491140 on chromosome 4 (genbank LT962479.1).

Specifically, the genetic modification is a deletion and/or insertion of one or more bases.

In a specific embodiment, the genetic modification is at least one point mutation.

In a further specific embodiment, the genetic modification is a deletion of 1 or more bases. Specifically, it is a deletion of 2, 3, 4, 5, 6, 7, 8, 9, 10, 11, 12, 13, 14, 15, 16, 17, 18, 19, 20, 25, 30, 35, 40, 45, 50, 60, 70, 80, 90, 100, 150, 200, 250, 300, 350, 400 or 500 nucleotides or more.

In yet a further specific embodiment, the deletion is a deletion of at least 50%, preferably at least 90%, of the gene at position 65654 on chromosome 2 (genbank LT962477.1), position 949930 on chromosome 1 (genbank LT962476.1), position 1303485 on chromosome 4 (genbank LT962479.1), position 1323758 on chromosome 1 (genbank LT962476.1) or position 1491140 on chromosome 4 (genbank LT962479.1).

In a specific embodiment, the genetic modification is an insertion or replacement of 1 or more bases. Specifically, it is an insertion or replacement of 2, 3, 4, 5, 6, 7, 8, 9, 10, 11, 12, 13, 14, 15, 16, 17, 18, 19, 20, 25, 30, 35, 40, 45, 50, 60, 70, 80, 90, 100, 150, 200, 250, 300, 350, 400 or 500 nucleotides or more.

In a further specific embodiment, the genetic modification is integration of the landing pad.

In a specific embodiment, the promoter comprised in the landing pad is a PDF or PDC promoter.

In a specific embodiment, the promoter comprised in the landing pad is a DAS1, DAS2, AOX1 or GAP (e.g. Qin et al, 2011) promoter. Further preferred promoters are for example the promoters as published by Vogl and Glieder 2013 and Vogl et al. 2016.

In a specific embodiment, the promoter comprised in the landing pad is a GCW14 (Liang et al. 2013), UPP (US20160097053A1) or pCS1 (U.S. Pat. No. 9,150,870B2) promoter.

In a specific embodiment, the promoter comprised in the landing pad is a bidirectional promoter.

In a further specific embodiment, the transcription terminator comprised in the landing pad is a pUC origin genetic element.

Specifically, the stuffer fragment has a length of at least 2, 3, 4, 5, 6, 7, 8, 9, 10, 11, 12, 13, 14, 15, 16, 17, 18, 19, 20, 25, 30, 35, 40, 45, 50, 60, 70, 80, 90, 100, 150, 200, 250, 300, 350, 400 or 500 nucleotides or more.

Further provided herein is a method of producing a recombinant Protein or Polypeptide of Interest (POI) comprising the steps of:
a. providing the genetically modified yeast cell comprising a landing pad as described herein,
b. replacing the stuffer fragment with a nucleic acid sequence encoding a POI, preferably using homologous recombination, or integrating the POI as an insertion at one of the homologous sequences
c. cultivating said genetically modified yeast cells in a culture medium under conditions that allow for expression of the POI, and
d. isolating the POI from the cells or the culture medium.

Further provided herein is the use of the genetically modified yeast cells described herein, for producing a recombinant protein or polypeptide of interest (POI). Specifically, the genetically modified yeast strains described herein are used for the production of a variety of different POIs.

### BRIEF DESCRIPTION OF DRAWINGS

FIG. 1 Transformation of Jackpot strain to other model protein producing strain by landing pad strategy based on homologous recombination.

FIG. 2 Expression of model protein 1 in platform strain LG2530 (in comparison to the wild type strain (BSYBG10). Relative comparison of protein activity. LG2530: parental Jackpot strain. Clones integrated in the LG2530 locus (patterned).

FIG. 3 Expression of model protein 1 in platform strain LG2531 (in comparison to the wild type strain (BSYBG11). Relative comparison of protein activity. LG2531: parental Jackpot strain.

FIG. 4 Expression of model protein 2 in platform strain LG2531 (in comparison to the wild type strain (BSYBG11). Relative comparison of protein activity. Clones integrated in the LG2531 locus are highlighted.

FIG. 5 Comparison of relative activity of Jackpot strain LG2531 and the newly discovered strain LG2532 (biological replicates 6).

FIG. 6 Expression of the Hypoxylon sp. UPO (OTA57433.1) under control of the P_{DF}, the P_{AOX1} and the P_{GAP} in the strain LG2531, termed “JP chassis”, and the wildtype strain BSYBG11. Expression was evaluated by determining ABTS activity in the cultivation supernatant of the respective expression strains.

FIG. 7 Amino acid sequences referred to herein.

### DESCRIPTION OF EMBODIMENTS

Unless indicated or defined otherwise, all terms used herein have their usual meaning in the art, which will be clear to the skilled person. Reference is for example made to the standard handbooks, such as Sambrook et al, “Molecular Cloning: A Laboratory Manual” (4th Ed.), Vols. 1-3, Cold Spring Harbor Laboratory Press (2012); Krebs et al., “Lewin's Genes XI”, Jones & Bartlett Learning, (2017), and Murphy & Weaver, “Janeway's Immunobiology” (9th Ed., or more recent editions), Taylor & Francis Inc, 2017.

The subject matter of the claims specifically refers to artificial products or methods employing or producing such artificial products, which may be variants of native (wild-type) products. Though there can be a certain degree of sequence identity to the native structure, it is well understood that the materials, methods and uses of the invention, e.g., specifically referring to isolated nucleic acid sequences, amino acid sequences, fusion constructs, expression constructs, transformed host cells and modified proteins, are “man-made” or synthetic, and are therefore not considered as a result of “laws of nature”.

The terms “comprise”, “contain”, “have” and “include” as used herein can be used synonymously and shall be understood as an open definition, allowing further members or parts or elements. “Consisting” is considered as a closest definition without further elements of the consisting definition feature. Thus “comprising” is broader and contains the “consisting” definition.

The term “about” as used herein refers to the same value or a value differing by +/−5% of the given value.

As used herein and in the claims, the singular form, for example “a”, “an” and “the” includes the plural, unless the context clearly dictates otherwise.

As used herein, amino acids refer to twenty naturally occurring amino acids encoded by sixty-one triplet codons. These 20 amino acids can be split into those that have neutral charges, positive charges, and negative charges:

The “neutral” amino acids are shown below along with their respective three-letter and single-letter code and polarity:
Alanine: (Ala, A) nonpolar, neutral;
Asparagine: (Asn, N) polar, neutral;
Cysteine: (Cys, C) nonpolar, neutral;
Glutamine: (Gln, Q) polar, neutral;
Glycine: (Gly, G) nonpolar, neutral;
Isoleucine: (Ile, I) nonpolar, neutral;
Leucine: (Leu, L) nonpolar, neutral;
Methionine: (Met, M) nonpolar, neutral;
Phenylalanine: (Phe, F) nonpolar, neutral;
Proline: (Pro, P) nonpolar, neutral;
Serine: (Ser, S) polar, neutral;
Threonine: (Thr, T) polar, neutral;
Tryptophan: (Trp, W) nonpolar, neutral;
Tyrosine: (Tyr, Y) polar, neutral;
Valine: (Val, V) nonpolar, neutral; and
Histidine: (His, H) polar, positive (10%) neutral (90%).

The “positively” charged amino acids are:
Arginine: (Arg, R) polar, positive; and
Lysine: (Lys, K) polar, positive.

The “negatively” charged amino acids are:
Aspartic acid: (Asp, D) polar, negative; and
Glutamic acid: (Glu, E) polar, negative.

The present disclosure is generally related to modified yeast cells producing increased amounts of one or more protein(s) of interest (hereinafter, a “POI”). Specifically, the present disclosure relates to modified yeast cells secreting one or more POI(s) with increased yield. Thus, certain embodiments of the instant disclosure are directed to modified yeast cells producing and/or secreting an increased amount of a POI relative to unmodified (parental) yeast cells producing and/or secreting the same POI, wherein the modified yeast cells comprise a modification at the specific sites described herein.

Surprisingly, the modified yeast cells of the present invention not only produce increased amounts of one POI, but the phenotype of enhanced expression is transferrable to other recombinant proteins or polypeptides.

As defined herein, a “modified cell”, a “modified yeast cell” or a “modified host cell” may be used interchangeably and refer to recombinant yeast (host) cells that comprise a modification (e.g., a genetic modification) which increases expression of the gene encoding the POI, also referred to as Gene of Interest (GOI). For example, a “modified” yeast cell of the instant disclosure may be further defined as a “modified (host) cell” which is derived from a parental yeast cell, wherein the modified (daughter) cell comprises a modification which increases GOI expression.

As defined herein, an “unmodified cell”, an “unmodified yeast cell” or an “unmodified host cell” may be used interchangeably and refer to “unmodified” (parental) yeast cells that do not comprise a modification at the specific sites described herein.

As used herein, when the expression and/or production and/or secretion of a POI in an “unmodified” (parental) cell is being compared to the expression and/or production and/or secretion of the same POI in a “modified” (daughter) cell, it will be understood that the “modified” and “unmodified” cells are grown/cultured/fermented under essentially the same conditions (e.g., the same conditions such as media, temperature, pH and the like).

Likewise, as defined herein, the terms “increased production” or “increased secretion”, “enhanced production”, “increased production of a POI”, “enhanced production of a POI”, and the like refer to a “modified” (daughter) cell comprising modification(s) as further described herein, wherein the “increase” is always relative (vis-à-vis) to an “unmodified” (parental) cell expressing and/or secreting the same POI.

The term “host cell” or “yeast cell” as referred to herein is understood as any yeast cell type that is susceptible to transformation, transfection, transduction, or the like with nucleic acid constructs or expression vectors comprising polynucleotides encoding expression products described herein, or susceptible to otherwise introduce any or each of the components of the fusion protein described herein. Specifically, the yeast cells referred to herein are of the genus Komagataella, and even more specifically of the species Komagataella phaffii (syn. Pichia pastoris). Specifically, the host yeast cells are maintained under conditions allowing expression of the POI.

The preferred yeast host cells are derived from methylotrophic yeast, such as from Pichia or Komagataella, e.g. Pichia pastoris , or Komagataella pastoris, or K. phaffii, or K. pseudopastoris. Examples of the host include yeasts such as P. pastoris. Examples of P. pastoris strains include CBS 704 (=NRRL Y-1603=DSMZ 70382), CBS 2612 (=NRRL Y-7556), CBS 7435 (=NRRL Y-11430, Wegner 21-1, ATCC 76273), CBS 9173-9189 (CBS strains: CBS-KNAW Fungal Biodiversity Centre, Centraalbureau voor Schimmelcultures, Utrecht, The Netherlands), and DSMZ 70877 (German Collection of Microorganisms and Cell Cultures), but also strains from Invitrogen, such as X-33, GS115, KM71 and SMD1168.

The term “cell line” as used herein refers to an established clone of a particular cell type that has acquired the ability to proliferate over a prolonged period of time. The term “host cell line” refers to a cell line as used for expressing an endogenous or recombinant gene or genes of interest to produce polypeptides or proteins of interest. A cell line prepared for recombination with one or more heterologous genes to incorporate the genes into the cells' genome, is herein also referred to as “chassis” cell line. A “production host cell line” or “production cell line” is commonly understood to be a cell line ready-to-use for cultivation/culturing in a bioreactor to obtain the product of a production process, such as a POI. The yeast host or yeast cell line as described herein is particularly understood as a recombinant yeast organism, which may be cultivated/cultured to produce a POI.

It has been surprisingly found that introducing in a yeast cell described herein, a genetic modification in the open reading frame at any one or more, or even all, of:
position 65654 on chromosome 2 (genbank LT962477.1),
position 949930 on chromosome 1 (genbank LT962476.1),
position 1303485 on chromosome 4 (genbank LT962479.1),
position 1323758 on chromosome 1 (genbank LT962476.1) and/or
position 1491140 on chromosome 4 (genbank LT962479.1)
   provides for increased production of a POI by the host cell.

As used herein, the term “position” refers to a genomic location, specifically:
position 949930 on chromosome 1 (genbank LT962476.1),
position 65654 on chromosome 2 (genbank LT962477.1),
position 1303485 on chromosome 4 (genbank LT962479.1),
position 1323758 on chromosome 1 (genbank LT962476.1) and/or
position 1491140 on chromosome 4 (genbank LT962479.1).

The numbering of the positions is according to the nucleic acid sequence as published under the respective genbank identifier, openly accessible e.g. under https://www.ncbi.nlm.nih.gov/genbank/

Specifically, the ORF, or the position itself, at any of
position 949930 on chromosome 1 (genbank LT962476.1),
position 65654 on chromosome 2 (genbank LT962477.1),
position 1303485 on chromosome 4 (genbank LT962479.1),
position 1323758 on chromosome 1 (genbank LT962476.1) and/or
position 1491140 on chromosome 4 (genbank LT962479.1) is also referred to herein as “integration site”. The integration site may comprise all or a part of the ORF, e.g. only the nucleobase at the position described herein or a sequence of a number of sequential bases comprising the nucleobase at the position described herein.

In a specific embodiment, the yeast cell described herein has a genetic modification as described herein in the gene located at the position selected from the group consisting of:
position 949930 on chromosome 1 (genbank LT962476.1),
position 65654 on chromosome 2 (genbank LT962477.1),
position 1303485 on chromosome 4 (genbank LT962479.1),
position 1323758 on chromosome 1 (genbank LT962476.1) and/or
position 1491140 on chromosome 4 (genbank LT962479.1).

Specifically, the yeast cell described herein has a genetic modification as described herein, specifically an inactivating mutation, in a gene selected from the group consisting of ALG9 (SEQ ID NO:14), SRB8 (SEQ ID NO:15), ACIB2EUKG772803 (SEQ ID NO:16), KAP123 (SEQ ID NO:17) and FLO400 (SEQ ID NO:18).

In a further specific embodiment, the yeast cell described herein has a genetic modification in every one of the genes at said positions. In yet a further specific embodiment, the yeast cell described herein comprises a genetic modification in 2, 3, or 4 of the genes at the positions described herein.

The genetic modification may be at any position within the gene(s), specifically within the open reading frame of the gene(s), located at the above-mentioned position(s).

The gene ALG9 is located on chromosome 2 and ranges from position 64999 to 66840 according to the numbering of the sequence of chromosome 2 published under genbank LT962477.1.

In a specific embodiment, the yeast cell described herein comprises a genetic modification as described herein in the ALG9 gene, specifically an inactivating modification. Specifically, the yeast cell described herein comprises a genetic modification as described herein within the wild type nucleotide sequence ranging from position 64999 to 66840 according to the numbering of the sequence of chromosome 2 published under genbank LT962477.1. Specifically, said modification is a mutation preventing expression of active native ALG9. Preferably, said modification prevents expression of full-length ALG9. Even more preferably, said modification prevents expression of ALG9. Specifically, said modification comprises, or consists of, a mutation at position 65654 on said chromosome 2.

ALG9 is a mannosyltransferase, involved in N-linked glycosylation. It is known to catalyze both the transfer of seventh mannose residue on B-arm and ninth mannose residue on the C-arm from Dol-P-Man to lipid-linked oligosaccharides.

In a specific embodiment, the yeast cell described herein comprises a genetic modification as described herein in the SRB8 gene, specifically an inactivating modification. Specifically, the yeast cell described herein comprises a genetic modification as described herein within the wild type nucleotide sequence ranging from position 945553 to 950169 according to the numbering of the sequence of chromosome 1 published under genbank LT962476.1. Specifically, said modification is a modification preventing expression of active SRB8. Preferably, said modification prevents expression of full-length SRB8. Even more preferably, said modification prevents expression of SRB8. Specifically, said modification comprises, or consists of, a mutation position 949930 on said chromosome 1.

SRB8 is a subunit of the RNA polymerase II mediator complex and is known to associate with core polymerase subunits to form the RNA polymerase II holoenzyme. SRB8 is known to be essential in S. cerevisiae for transcriptional regulation.

In a specific embodiment, the yeast cell described herein comprises a genetic modification as described herein in the ACIB2EUKG772803 gene, specifically an inactivating modification. Specifically, the yeast cell described herein comprises a genetic modification as described herein within the wild type nucleotide sequence ranging from position 1301453 to 1303573 according to the numbering of the sequence of chromosome 4 published under genbank

LT962479.1. Specifically, said modification is a modification preventing expression of active ACIB2EUKG772803. Preferably, said modification prevents expression of full-length ACIB2EUKG772803. Even more preferably, said modification prevents expression of ACIB2EUKG772803. Specifically, said modification comprises, or consists of, a mutation position 1303485 on said chromosome 4.

ACIB2EUKG772803 is a ferric reductase, known to reduce siderophore-bound iron prior to uptake by transporters.

The gene FLO400 is located on chromosome 4 of Komagataella phaffii.

In a specific embodiment, the yeast cell described herein comprises a genetic modification as described herein in the FLO400 gene, specifically an inactivating modification. Specifically, the yeast cell described herein comprises a genetic modification as described herein within the wild type nucleotide sequence comprising or consisting of SEQ ID NO:18. Specifically, said modification is a mutation preventing expression of active native FLO400. Preferably, said modification prevents expression of full-length FLO400. Even more preferably, said modification prevents expression of FLO400. Specifically, said modification comprises, or consists of, a mutation at position 1491140 on said chromosome 4, the sequence of which is published under genbank LT962479.1.

The gene KAP123 is located on chromosome 1 of Komagataella phaffii.

In a specific embodiment, the yeast cell described herein comprises a genetic modification as described herein in the KAP123 gene, specifically an inactivating modification. Specifically, the yeast cell described herein comprises a genetic modification as described herein within the wild type nucleotide sequence comprising or consisting of SEQ ID NO:17. Specifically, said modification is a mutation preventing expression of active native KA123. Preferably, said modification prevents expression of full-length KAP123. Even more preferably, said modification prevents expression of KAP123. Specifically, said modification comprises, or consists of, a mutation at position 1323758 on said chromosome 1, according to the numbering of the sequence of chromosome 1 published under genbank LT962476.1.

As used herein, the term “genetic modification” refers to any change within a nucleotide sequence which results in the addition, deletion, or alteration, specifically substitution, of at least one nucleotide. Specifically, the genetic modification may be any of a mutation, insertion or deletion, or any combination thereof. In a specific aspect, the genetic modification results in a change of the open reading frame.

In a preferred embodiment, the genetic modification described herein is a loss-of-function mutation, also called “inactivating mutation” or “inactivating modification”, which results in the gene product having less or no function, i.e. being partially or wholly inactivated. Preferably, the resulting gene product, i.e. polypeptide, has no function. An inactivating mutation may also result in no gene product being produced.

In a specific embodiment, the genetic modification is an insertion of more than one nucleotide, specifically it is an insertion of a nucleotide sequence, specifically an insertion of an oligonucleotide or a polynucleotide. In a specific aspect, such nucleotide sequence is an expression cassette, such as the landing pad described herein, or a gene of interest, preferably comprised in an expression cassette. In another specific aspect, the inserted nucleotide sequence inserted is a random sequence.

In a further specific embodiment, the genetic modification is a deletion of more than one nucleotide. Specifically, it is a deletion of part or all of the gene(s) at the position(s) described herein. Specifically, it is a deletion of at least one exon of the gene(s) at the position(s) described herein.

As used herein, the term “mutation” has its ordinary meaning in the art. A mutation may comprise a point mutation, or refer to areas of sequences, in particular changing contiguous or non-contiguous amino acid sequences. Specifically, a mutation is a point mutation, which is herein understood as a mutation to alter one or more (but only a few) contiguous nucleotides or amino acids, e.g. 1, or 2, or 3 nucleotides or amino acids are substituted, inserted or deleted at one position in an amino acid sequence. Amino acid substitutions may be conservative amino acid substitutions or non-conservative amino acid substitutions. Conservative substitutions, as opposed to non-conservative substitutions, comprise substitutions of amino acids belonging to the same set or sub set, such as hydrophobic, polar, etc. Point mutations in a nucleic acid sequence may specifically include frameshift mutations that disrupt gene function or gene expression (gene knock-outs).

The terms “polynucleotide,” “oligonucleotide,” “nucleic acid” and “nucleic acid molecule” and “gene” are used interchangeably herein to refer to a polymeric form of nucleotides of any length, and may comprise ribonucleotides, deoxyribonucleotides, analogs thereof, or mixtures thereof. This term refers only to the primary structure of the molecule. Thus, the term includes triple-, double- and single-stranded deoxyribonucleic acid (“DNA”), as well as triple-, double and single-stranded ribonucleic acid (“RNA”). It also includes modified, for example by alkylation, and/or by capping, and unmodified forms of the polynucleotide. More particularly, the terms “polynucleotide,” “oligonucleotide,” “nucleic acid” and “nucleic acid molecule” include polydeoxyribonucleotides (containing 2-deoxy-D-ribose), polyribonucleotides (containing D-ribose), including tRNA, rRNA, hRNA, siRNA and mRNA.

The terms “polynucleotide,” “oligonucleotide,” “nucleic acid” and “nucleic acid molecule” and “gene” refer to the entire sequence or gene or a fragment thereof. The fragment thereof can be a functional fragment. Where the polynucleotides are to be used to express encoded proteins, nucleotides that can perform that function or which can be modified (for example, reverse transcribed) to perform that function are used. Where the polynucleotides are to be used in a scheme that requires that a complementary strand be formed to a given polynucleotide, nucleotides are used which permit such formation.

As used herein, the terms “nucleoside” and “nucleotide” will include those moieties which contain not only the known purine and pyrimidine bases, but also other heterocyclic bases which have been modified. Such modifications include methylated purines or pyrimidines, acylated purines or pyrimidines, or other heterocycles. Modified nucleosides or nucleotides can also include modifications on the sugar moiety, for example, where one or more of the hydroxyl groups are replaced with halogen, aliphatic groups, or is functionalized as ethers, amines, or the like.

It is understood that the polynucleotides (or nucleic acid molecules) described herein include “genes”, “vectors” and “plasmids”. Accordingly, the term “gene”, refers to a polynucleotide that codes for a particular sequence of amino acids, which comprise all, or part of a protein coding sequence, and may include regulatory (non-transcribed) DNA sequences, such as promoter sequences, which determine for example the conditions under which the gene is expressed. The transcribed region of the gene may include untranslated regions (UTRs), including introns, 5′-untranslated region (UTR), and 3′-UTR, as well as the coding sequence.

As used herein, the term “coding sequence” refers to a nucleotide sequence, which directly specifies the amino acid sequence of its (encoded) protein product. The boundaries of the coding sequence are generally determined by an open reading frame, which usually begins with an ATG start codon. The coding sequence typically includes DNA, cDNA, and recombinant nucleotide sequences.

“Open Reading Frame” or “ORF” means a portion of a DNA molecule that, when translated into amino acids, contains no stop codons. Specifically, the term “open reading frame” (hereinafter, “ORF”) means a nucleic acid or nucleic acid sequence (whether naturally occurring, non-naturally occurring, or synthetic) comprising an uninterrupted reading frame consisting of (i) an initiation codon, (ii) a series of two (2) or more codons representing amino acids, and (iii) a termination codon, the ORF being read (or translated) in the 5′ to 3′ direction. The genetic code reads DNA sequences in groups of three base pairs, which means that a double-stranded DNA molecule can read in any of six possible reading frames-three in the forward direction and three in the reverse. A long open reading frame is likely a part of a gene. For example, the yeast cell described herein comprises a modification in the ORF encoding ALG9, SRB8 and/or ACIB2EUKG772803.

The term “Protein of Interest” (POI) as used herein refers to a polypeptide or a protein that is produced by means of recombinant technology in a host cell. More specifically, the protein may either be a polypeptide not naturally occurring in the host cell, i.e. a heterologous protein, or else may be native to the host cell, i.e. a homologous protein to the host cell, but is produced, for example, by transformation with a self-replicating vector containing the nucleic acid sequence encoding the POI, or upon integration by recombinant techniques of one or more copies of the nucleic acid sequence encoding the POI into the genome of the host cell, or by recombinant modification of one or more regulatory sequences controlling the expression of the gene encoding the POI, e.g. of a promoter sequence.

The POI can be any eukaryotic, prokaryotic or synthetic polypeptide. Specifically, it can be a mammalian protein, including human or animal proteins. It can be a secreted protein or an intracellular protein. A POI can be a naturally occurring protein, or an artificial protein. The present methods and yeast host cells are also provided for the recombinant production of functional variants, derivatives or biologically active fragments of naturally occurring proteins.

A POI referred to herein may be a product homologous (or allogenic) to the eukaryotic host cell or a heterologous one, and is preferably prepared for therapeutic, prophylactic, diagnostic, analytic or industrial use.

The POI is preferably a heterologous recombinant polypeptide or protein, produced in a yeast cell. The POI may be produced as intracellular or as secreted proteins. Examples of preferably produced proteins are enzymes, regulatory proteins, receptors, peptides, e.g. peptide hormones, cytokines, structural proteins, e.g. collagen, spider silks, or other proteins such as milk proteins, whey proteins, food and feed additive proteins, serum albumin proteins, and membrane or transport proteins. The proteins of interest may also be antigens as used for vaccination (e.g. viral envelope proteins), vaccines, antigen-binding proteins, immune stimulatory proteins, allergens, full-length antibodies or antibody fragments or derivatives. Antibody derivatives may be for example single chain variable fragments (scFv), Fab fragments or single domain antibodies.

The POI may be a protein that is structurally similar to a native protein and may be derived from the native protein by addition of one or more amino acids to either or both the C- and N-terminal end or the side-chain of the native protein, substitution of one or more amino acids at one or a number of different sites in the native amino acid sequence, deletion of one or more amino acids at either or both ends of the native protein or at one or several sites in the amino acid sequence, or insertion of one or more amino acids at one or more sites in the native amino acid sequence.

A POI can also be selected from substrates, enzymes, inhibitors or cofactors that provide for biochemical reactions in the host cell, with the aim to obtain the product of said biochemical reaction or a cascade of several reactions, e.g. to obtain a metabolite of the host cell. Exemplary products can be vitamins, such as riboflavin, organic acids, and alcohols, which can be obtained with increased yields following the expression of a recombinant protein or a POI described herein.

The DNA molecule encoding the protein of interest is also termed “Gene of Interest” or “GOI”. The gene of interest encoding the POI can be a naturally existing DNA sequence or a non-natural DNA sequence. One or more gene of interests can be under the control of one promoter as described herein. Alternatively, each gene of interest is under one promoter. The gene of interests may all be on the same expression cassette or on multiple expression cassettes. The POI can be modified in any way. Non-limiting examples for modifications can be insertion or deletion of post-translational modification sites, insertion or deletion of targeting signals (e.g.: leader peptides), fusion to tags, proteins or protein fragments facilitating purification or detection, mutations affecting changes in stability or changes in solubility or any other modification known in the art. In certain embodiments of the invention the recombinant protein is a biopharmaceutical product, which can be any protein suitable for therapeutic or prophylactic purposes in mammals.

The term “functional variant” or “functionally active variant” also includes naturally occurring allelic variants, as well as mutants or any other non-naturally occurring variants. As is known in the art, an allelic variant is an alternate form of a nucleic acid or peptide that is characterized as having a substitution, deletion, or addition of one or nucleotides or more amino acids that does essentially not alter the biological function of the nucleic acid or polypeptide.

Functional variants may be obtained by sequence alterations in the polypeptide or the nucleotide sequence, e.g. by one or more point mutations, wherein the sequence alterations retain or improve a function of the unaltered polypeptide or the nucleotide sequence, when used in combination of the invention. Such sequence alterations can include, but are not limited to, (conservative) substitutions, additions, deletions, mutations and insertions. Conservative substitutions are those that take place within a family of amino acids that are related in their side chains and chemical properties. Examples of such families are amino acids with basic side chains, with acidic side chains, with non-polar aliphatic side chains, with non-polar aromatic side chains, with uncharged polar side chains, with small side chains, with large side chains etc.

The terms “heterologous” or “recombinant” as used herein with respect to a nucleotide sequence, construct such as an expression cassette, amino acid sequence or protein, refers to a compound which is either foreign to a given host cell, i.e. “exogenous”, such as not found in nature in said host cell; or that is naturally found in a given host cell, e.g., is “endogenous”, however, in the context of a heterologous construct or integrated in such heterologous construct, e.g., employing a heterologous nucleic acid fused or in conjunction with an endogenous nucleic acid, thereby rendering the construct heterologous, thus “not naturally-occurring”. The heterologous nucleotide sequence as found endogenously may also be produced in an unnatural, e.g., greater than expected or greater than naturally found, amount in the cell. The heterologous nucleotide sequence, or a nucleic acid comprising the heterologous nucleotide sequence, possibly differs in sequence from the endogenous nucleotide sequence but encodes the same protein as found endogenously. Specifically, heterologous nucleotide sequences are those not found in the same relationship to a host cell in nature (i.e., “not natively associated”). Any recombinant or artificial nucleotide sequence is understood to be heterologous.

Specifically, the term “recombinant” as used herein shall mean “being prepared by or the result of genetic engineering”. Thus, a “recombinant microorganism” comprises at least one “recombinant nucleic acid”. The yeast described herein is understood as a recombinant yeast. A recombinant microorganism may comprise an expression vector or cloning vector, or it has been genetically engineered to contain a recombinant nucleic acid sequence.

A “recombinant protein” is produced by expressing a respective recombinant nucleic acid in a host. A “recombinant promoter” is a genetically engineered non-coding nucleotide sequence suitable for its use as a functionally active promoter as described herein.

In general, the recombinant nucleic acids or organisms as referred to herein may be produced by recombination techniques well known to a person skilled in the art. In accordance with the present invention there may be employed conventional molecular biology, microbiology, and recombinant DNA techniques within the skill of the art. Such techniques are explained fully in the literature. See, e.g., Maniatis, Fritsch & Sambrook, “Molecular Cloning: A Laboratory Manual, Cold Spring Harbor, (1982).

According to a specific embodiment described herein, a recombinant construct is prepared by ligating a promoter and relevant gene(s) encoding a POI into a vector or expression construct. The gene(s) can be stably integrated into the host cell genome by transforming the host cell using vectors or expression constructs comprising an expression cassette that is integrated into the host genome using e.g. homologous recombination. The GOI can also be integrated into the host genome for transient expression using, e.g. self-replicating plasmids comprising the GOI.

In a preferred embodiment, the GOI is stably integrated in the yeast genome, e.g. by homologous recombination.

The yeast cell may also comprise the GOI on an extrachromosomal genetic element. In a specific embodiment, the GOI is comprised on a plasmid, as further described herein. According to a specific example, the GOI may be expressed from a centromer-based plasmid. According to a further specific example, the GOI may be comprised on an artificial chromosome.

Integration of one or more recombinant genes into the genome results in a discrete and pre-defined number of genes of interest per cell. In the embodiment of the invention that inserts one copy of the gene, this number is usually one (except in the case that a cell contains more than one chromosome or genome, as it occurs transiently during cell division), as compared to plasmid-based expression which is accompanied by copy numbers up to several hundred. In the expression system used in the method of the present invention, by relieving the host metabolism from plasmid replication, an increased fraction of the cells' synthesis capacity is utilized for recombinant protein production.

In view of site-specific gene insertion, another requirement to the host cell is that it contains at least one genomic region (either a coding or any non-coding functional or non-functional region or a region with unknown function) that is known by its sequence and that can be disrupted or otherwise manipulated to allow insertion of a heterologous sequence, without being detrimental to the cell. As described herein, introducing a genetic modification at the integration sites described herein allows improved POI production. It is thus a particularly advantageous aspect of the present invention, to introduce an empty cassette, also referred to herein as landing pad, at the integration sites described herein. Thereby, expression of the native gene at the respective site is disrupted and POI expression is enhanced as compared to POI expression in a yeast cell not comprising a genetic modification at the integration site and where the POI is integrated into the genome at a site differing from the sites described herein.

With regard to the integration locus, the expression system used in the invention allows for a wide variability. In principle, any locus with known sequence may be chosen, with the proviso that the function of the sequence is either dispensable or, if essential, can be complemented (as e.g. in the case of an auxotrophy) and that the yeast cell in addition comprises a genetic modification at the sites described herein.

Integration of the gene of interest into the yeast genome can be achieved by conventional methods, e.g. transformation of a yeast cell by using linear DNA constructs (expression cassettes) that contain flanking sequences homologous to a specific site on the chromosome, also called homologous recombination. Moreover, the use of a linear expression cassette provides the advantage that the genomic integration site can be freely chosen by the respective design of the flanking homologous regions of the cassette. Thereby, integration of the linear expression cassette allows for greater variability with regard to the genomic region. The integration method used herein is not limited to the above-mentioned example; rather any integration method known in the art can be used.

The integration methods for obtaining the host cell are not limited to integration of one gene of interest at one site in the genome; they allow for variability with regard to both the integration site and the expression cassettes. By way of example, more than one gene of interest may be inserted, i.e. two or more identical or different GOIs under the control of identical or different promoters can be integrated into one or more different loci on the genome. By way of example, it allows expression of two different proteins that form a heterodimeric complex. Heterodimeric proteins consist of two individually expressed protein subunits, e.g. the heavy and the light chain of a monoclonal antibody or an antibody fragment.

In a specific embodiment, the GOI is introduced into the genome of the host cell via homologous recombination. “Homologous recombination” refers to a reaction between nucleotide sequences having corresponding sites containing a similar nucleotide sequence (i.e., homologous sequences) through which the molecules can interact (recombine) to form a new, recombinant nucleic acid sequence. The sites of similar nucleotide sequences are each referred to herein as a “homologous sequence”. Generally, the frequency of homologous recombination increases as the length of the homology sequence increases. Thus, while homologous recombination can occur between two nucleic acid sequences that are less than identical, the recombination frequency (or efficiency) declines as the divergence between the two sequences increases.

Recombination may be accomplished using one homology sequence on each of two molecules to be combined, thereby generating a “single-crossover” recombination product. Alternatively, two homology sequences may be placed on each of two molecules to be recombined. Recombination between two homology sequences on the donor with two homology sequences on the target generates a “double-crossover” recombination product.

Therefore, in order for two polynucleotide sequences to be recombined by homologous recombination with each other, both polynucleotides need to share a region of homology with each other. These regions of homology are called interchangeably herewith “flanking regions”, “flanking sequences”, “overlapping regions”, “overlapping sequences”, “homologous regions”, “homologous sequences”. In order for homologous recombination to take place the homologous sequences do not need to be identical. However, the efficiency of homologous recombination increases with the level of sequence identity between the homologous sequences. Preferably the homologous sequence will be at least 50% identical, preferably at least 60%, 70%, 80%, 85%, 90%, 95%, identical with each other, more preferably the homologous sequences will be 100% identical with each other. It is known to those skilled in the art that efficiency of homologous recombination increases with the length of the homologous sequences between the polynucleotides to be recombined. In one embodiment the homologous sequences are at least 10 bp long, preferably at least 20 bp, 30 bp, 40 bp, 50 bp, 100 bp, 500 bp, 1000 bp or more.

The term “recognition sequence” or “target sequence” refers to particular DNA sequences which are recognized (and bound by) a protein, DNA, or RNA molecule, including a restriction endonuclease, a modification methylase, and a recombinase. For example, the recognition sequence for Cre recombinase is loxP which is a 34 base pair sequence comprised of two 13 base pair inverted repeats (serving as the recombinase binding sites) flanking an 8 base pair core sequence. Other examples of recognition sequences are the attB, attP, attL, and attR sequences which are recognized by the integrase of bacteriophage lambda and the FRT recognition sequence which is recognized by Flp (Flippase). AttB is an approximately 25 base pair sequence containing two 9 base pair core-type Int binding sites and a 7 base pair overlap region. attP is an approximately 240 base pair sequence containing core-type Int binding sites and arm-type Int binding sites as well as sites for auxiliary proteins IHF, FIS, and Xis. Such sites are also engineered according to the present invention to enhance methods and products. The term “Recombinase” refers to an enzyme which catalyzes the exchange of DNA segments at specific recombination sites. The term “Recombinational Cloning” refers to a method whereby segments of DNA molecules are exchanged, inserted, replaced, substituted or modified, in vitro or in vivo. The term “Recombination proteins” includes excisive or integrative proteins, enzymes, co-factors or associated proteins that are involved in recombination reactions involving one or more recombination sites.

The term “selection marker” refers to a polynucleotide segment that allows one to select for or against a molecule or a cell that contains it, often under particular conditions. These markers can encode an activity, such as, but not limited to, production of RNA, peptide, or protein, or can provide a binding site for RNA, peptides, proteins, inorganic and organic compounds or compositions. Examples of selectable markers include but are not limited to: (1) DNA segments that encode products which provide resistance against otherwise toxic compounds (e.g., antibiotics); (2) DNA segments that encode products which are otherwise lacking in the recipient cell (e.g., tRNA genes, auxotrophic markers); (3) DNA segments that encode products which suppress the activity of a gene product; (4) DNA segments that encode products which can be readily identified (e.g., phenotypic markers such as beta-galactosidase, green fluorescent protein (GFP), red fluorescent protein (RFP), and cell surface proteins); (5) DNA segments that bind products which are otherwise detrimental to cell survival and/or function; (6) DNA segments that otherwise inhibit the activity of any of the DNA segments described in Nos. 1-5 above (e.g., antisense oligonucleotides); (7) DNA segments that bind products that modify a substrate (e.g. restriction endonucleases); (8) DNA segments that can be used to isolate a desired molecule (e.g. specific protein binding sites); (9) DNA segments that encode a specific nucleotide sequence which can be otherwise non-functional (e.g., for PCR amplification of subpopulations of molecules); and/or (10) DNA segments, which when absent, directly or indirectly confer sensitivity to particular compounds; and/or DNA segments that encode a gene coding for a protein which enables the utilization of a specific carbon or nitrogen source.

Further specific examples of selection markers include antibiotic resistance genes such as Geneticin/Kanamycin marker (KanMX), Zeocin or Hygromycin marker, nourseothricin.

Further specific examples of selection markers include auxotrophic selection markers such as for example based on the HIS4, URA3, ARG4, MET1, ADE1, ADE2, ADE3 genes, or carbon source utilization selection markers such as glycerol kinase (GUT1), AOX1/AOX2, TPI, or DAS1&DAS2, or other nutrient utilization markers such as nitrogen utilization based on the AMDS gene. Alternatively, fluorinated substrate derivatives such as 5FOA or fluoracetamide can be used for counterselection.

A “reporter gene” typically encodes a protein, also referred to as “reporter protein”, the expression of which can readily be detected. A typical example of a reporter protein is a fluorescent protein, such as e.g. GFP, RFP, YFP or mCherry, which can be directly detected or an enzyme reporter such as β-galactosidase or β-glucuronidase or luciferase which can be detected by colorimetric, fluorimetric or chemoluminescence assays. In a specific embodiment, the stuffer fragment of the landing pad described herein comprises or consists of a reporter gene. Upon replacement of the stuffer fragment with the GOI, the lack of expression of the reporter protein indicates successful replacement of the stuffer fragment with the GOI.

The term “expression” as used herein regarding expressing a polynucleotide or nucleotide sequence, is meant to encompass at least one step selected from the group consisting of DNA transcription into mRNA, mRNA processing, non-coding mRNA maturation, mRNA export, translation, protein folding and/or protein transport. Nucleic acid molecules containing a desired nucleotide sequence may be used for producing an expression product encoded by such nucleotide sequence e.g., proteins or polypeptides of interest as described herein. To express a desired nucleotide sequence, an expression system is conveniently used, which can be an in vitro or in vivo expression system, as necessary to express a certain nucleotide sequence by a host cell or host cell line. Typically, host cells are transfected or transformed with an expression system comprising an expression cassette that comprises the desired nucleotide sequence and a promoter operably linked thereto optionally together with further expression control sequences or other regulatory sequences. Coding DNA is a DNA sequence that encodes a particular amino acid sequence for a particular polypeptide or protein. Promoter DNA is a DNA sequence which initiates, regulates, or otherwise mediates or controls the expression of the coding DNA. Promoter DNA and coding DNA may be from the same gene or from different genes, and may be from the same or different organisms. Recombinant cloning vectors often include one or more replication systems for cloning or expression, one or more markers for selection in the host, e.g., antibiotic resistance, one or more nuclear localization signals (NLS) and one or more expression cassettes.

Specific expression systems employ expression constructs such as vectors comprising one or more expression cassettes.

The term “expression construct” as used herein, means the vehicle, e.g. vectors or plasmids, by which a DNA sequence is introduced into a host cell so as to transform the host and promote expression (e.g. transcription and translation) of the introduced sequence. “Expression construct” as used herein includes both, autonomously replicating nucleotide sequences as well as genome integrating nucleotide sequences.

The terms “vector”, “DNA vector” and “expression vector” mean the vehicle by which a DNA sequence (e.g. a foreign gene) can be introduced into a host cell, so as to transform the host and promote expression (e.g. transcription and translation) of the introduced sequence. “Vector” as used herein includes both, autonomously replicating nucleotide sequences as well as genome integrating nucleotide sequences, such as artificial chromosomes. A common type of vector is a “plasmid”, which generally is a self-contained molecule of double-stranded DNA that can readily accept additional (foreign) DNA and which can readily be introduced into a suitable host cell. Specifically, the term “vector” or “plasmid” refers to a vehicle by which a DNA or RNA sequence (e.g. a foreign gene) can be introduced into a host cell, so as to transform the host and promote expression (e.g. transcription and translation) of the introduced sequence.

The term “expression vector” means a linear or circular DNA molecule that comprises a polynucleotide encoding a polypeptide and is operably linked to control sequences that provide for its expression. Vectors typically comprise DNA sequences that are required for the transcription of cloned recombinant nucleotide sequences, i.e. of recombinant genes and the translation of their mRNA in a suitable host organism. A coding DNA sequence or segment of DNA molecule coding for an expression product can be conveniently inserted into a vector at defined restriction sites. To produce a vector, heterologous foreign DNA can be inserted at one or more restriction sites of a vector DNA, and then is carried by the vector into a host cell along with the transmissible vector DNA. It is preferred that a vector comprises an expression system, e.g. one or more expression cassettes. Expression cassette restriction sites are designed to ensure insertion of the cassette in the proper reading frame.

To obtain expression, a sequence encoding a desired expression product, such as e.g. any of the polypeptides, proteins or protein domains described herein, is typically cloned into an expression vector that contains a promoter to direct transcription. Appropriate expression vectors typically comprise regulatory sequences suitable for expressing coding DNA. Examples of regulatory sequences include promoter, operators, enhancers, ribosomal binding sites, and sequences that control transcription and translation initiation and termination. The regulatory sequences are typically operably linked to the DNA sequence to be expressed.

A promoter is herein understood as a DNA sequence which initiates, regulates, or otherwise mediates or controls the expression of the coding DNA. Specifically, “promoter” refers to a DNA sequence capable of controlling the expression of a coding sequence or functional RNA. Promoter DNA and coding DNA may be from the same gene or from different genes, and may be from the same or different organisms. Promoter activity may be assessed by its transcriptional efficiency. This may be determined directly by measurement of the amount of mRNA transcription from the promoter, e.g. by Northern Blotting or indirectly by measurement of the amount of gene product expressed from the promoter.

In a specific embodiment, the promoter is a derepressible promoter, preferably selected from the group consisting of the PDC promoter and PDF promoter, as described for example in Fischer et al. 2019, PDF promoter variants, Hansenula polymorpha FMD promoter, Hansenula polymorpha MOX1 promoter P. pastoris Catalase 1 promoter, PEX promoter, P pastoris FMD promoter or a synthetic promoter generated by fusion upstream of core promoter sequences with derepressible regulator sequence, or active variants thereof.

In a further specific embodiment, the promoter is an inducible promoter, preferably selected from the group consisting of AOX1 promoter, DAS1 or DAS2 promoter, PGK promoter, ADH promoter, FMD promoter, GTH1 promoter, FDH promoter and FLD promoter, or active variants thereof, or inducible synthetic or orthologous promoters from other organisms such as a GAL or LAC promoter.

In yet a further specific embodiment, the promoter is a constitutive promoter, preferably selected from the group consisting of GAP promoter, AOD1 promoter, HTA or HTX histone promoters, GCW14 promoter, PGK promoter, TEF1 promoter or active variants thereof or synthetic constitutive promoters made by fusions of core promoter elements with positive or negative regulatory DNA elements.

As an alternative to native or wild-type promoter sequences, functional variants of such native or wild-type promoter sequences (herein understood as parent promoters) can be used, which have at least 90% sequence identity and are functional in controlling the expression of a gene in substantially similar way, e.g. being an inducible promoter or constitutive promoter as the parent promoter.

The term “operably linked” as used herein refers to the association of nucleotide sequences on a single nucleic acid molecule, i.e. the vector, in a way such that the function of one or more nucleotide sequences is affected by at least one other nucleotide sequence present on said nucleic acid molecule. For example, a promoter is operably linked with a coding sequence encoding the protein of interest, when it is capable of effecting the expression of that coding sequence. Specifically, such nucleic acids operably linked to each other may be immediately linked, i.e. without further elements or nucleic acid sequences in between or may be indirectly linked with spacer sequences or other sequences in between.

A promoter sequence is typically understood to be operably linked to a coding sequence, if the promoter controls the transcription of the coding sequence. If a promoter sequence is not natively associated with the coding sequence, its transcription is either not controlled by the promoter in native (wild-type) cells or the sequences are recombined with different contiguous sequences.

Recombinant cloning vectors often include one or more replication systems for cloning or expression, one or more markers for selection in the host, e.g., antibiotic resistance, one or more localization signals (Sig) and one or more expression cassettes.

In specific embodiments, an expression vector may contain more than one expression cassette, each comprising at least one coding sequence and a promoter in operable linkage.

A “cassette” or “expression cassette” refers to a DNA coding sequence or segment of DNA that codes for an expression product. Typically, foreign DNA is inserted at one or more restriction sites of the vector DNA, and then is transferred by the vector into a host cell along with the transmissible vector DNA. A segment or sequence of DNA having inserted or added DNA, such as an expression vector, can also be called a “DNA construct”.

The term “expression cassette” or “cassette” as used herein refers to nucleic acid molecules containing a desired coding sequence and control sequences in operable linkage, so that an expression system can use such expression cassettes to produce the respective expression products, including e.g., encoded proteins or other expression products. Certain expression systems employ host cells or host cell lines which are transformed or transfected with an expression cassette, which host cells are then capable of producing expression products in vivo. In order to effect transformation of host cells, an expression cassette may be conveniently included in a vector, which is introduced into a host cell; however, the relevant DNA may also be integrated into a host chromosome.

The terms “expression cassette”, or simply “cassette”, synonymously used with “expression cartridge” or simply “cartridge”, refer to a linear or circular DNA construct to be integrated into the genome, such as a eukaryotic genome. As a result of integration, the expression host cell has an integrated expression cassette. Preferably, the cassette is a linear DNA construct comprising essentially a promoter, a gene of interest, immediately upstream of the gene of interest a potential Kozak consensus sequence, and two terminally flanking regions which are homologous to a genomic region and which enable homologous recombination. The cassette also may contain a bacterial promoter sequence and a ribosome binding site (RBS or SD) in the 5′ UTR of the region coding for the POI, which enable transcription by prokaryotes and which can serve as landing pad sequences for site specific integration in eukaryotic genomes. In addition, the cassette may contain other sequences such as for example sequences coding for antibiotic selection markers, prototrophic selection markers or fluorescent markers, markers coding for a metabolic gene, genes which improve protein expression or two flippase recognition target sites (FRT) which enable the removal of certain sequences (e.g. antibiotic resistance genes) after integration.

The expression cassette is synthesized and amplified by methods known in the art, in the case of linear cassettes, usually by standard polymerase chain reaction, PCR. Since linear cassettes are usually easier to construct, they are preferred for obtaining the expression host cells used in the system and method provided herein. Moreover, the use of a linear expression cassette provides the advantage that the genomic integration site can be freely chosen by the respective design of the flanking homologous regions of the cassette. Thereby, integration of the linear expression cassette allows for greater variability with regard to the genomic region.

The term “landing pad” as used herein refers to a heterologous sequence in the host cell genome comprising target sequences for site-specific integration of a gene of interest. Specifically, the term “landing pad” refers to an empty expression cassette comprising target sequences for homologous recombination. In a specific embodiment, the empty expression cassette comprises genetic elements typically required for expression of a POI but does not comprise the sequence encoding the POI. Instead, the empty expression cassette may comprise a stuffer fragment as described herein. In a specific embodiment, the landing pad comprises any one or more of a selection marker or reporter protein, a stuffer fragment, a promoter 5′ of said stuffer fragment and a transcription terminator 3′ of said stuffer fragment.

In a specific embodiment, the target sequences for homologous recombination of the landing pad described herein comprise sequences typical of an expression cassette, such as for example origin of replication and promoter sequences, e.g. pUCORI and/or PDF promoter sequence.

In a specific embodiment, the landing pad is located at one or more of the integration sites described herein. Specifically, the target sequences of the landing pad within the integration site(s) described herein comprise the nucleic acid sequence about 1 kb upstream and downstream of the ORF at the positions described herein. Specifically, the target sequence is a homologous genomic region comprising about 0,3-3 kb, preferably about 1 kb, upstream and downstream of the 5′ and 3′ untranslated and translated region of the genes at the positions described herein. The genomic region can be a native sequence of the host genome or a modified sequence providing a preferred landing pad sequence.

Expression vectors may comprise the expression cassette described herein and in addition optionally comprise flanking regions homologous to the genome integration site, a number of restriction enzyme cleavage sites, an initial transcribed sequence (ITS) and a polyadenylation site and a transcription terminator, and optionally one or more selectable markers (e.g., an amino acid synthesis gene or a gene conferring resistance to antibiotics such as zeocin, kanamycin, geneticin, hygromycin, phleomycin or nourseothricin), which components are operably linked together.

Expression products such as polypeptides-, proteins- or protein domains-, of interest, as described herein may be introduced into a host cell either by introducing the respective coding polynucleotide or nucleotide sequence for expressing the expression products within the host cell, or by introducing the respective expression products which are within an expression system or isolated.

Any of the known procedures for introducing expression cassettes, vectors or otherwise introducing (e.g., coding) nucleotide sequences into host cells may be used. These include the use of calcium phosphate transfection, polybrene, protoplast fusion, electroporation, nucleofection, liposomes, microinjection, naked DNA, plasmid vectors, viral vectors, both episomal and integrative, and any of the other well-known methods for introducing cloned genomic DNA, cDNA, synthetic DNA or other foreign genetic material into a host cell (see, e.g., Sambrook et al.).

Expression vectors may include but are not limited to cloning vectors, modified cloning vectors and specifically designed plasmids. Any expression vector suitable for expression of a recombinant gene in a host cell can be used. Such vectors are typically selected depending on the host organism.

Appropriate expression vectors typically comprise further regulatory sequences suitable for expressing DNA encoding a POI in a yeast host cell. Examples of regulatory sequences include operators, enhancers, ribosomal binding sites, and sequences that control transcription and translation initiation and termination. The regulatory sequences may be operably linked to the DNA sequence to be expressed.

To allow expression of a recombinant nucleotide sequence in a host cell, the expression vector may provide the promoter adjacent to the 5′ end of the coding sequence, e.g. upstream from a gene of interest or a signal peptide gene enabling secretion of a POI. The transcription is thereby regulated and initiated by this promoter sequence.

The term “signal peptide” as used herein shall specifically refer to a native signal peptide, a heterologous signal peptide or a hybrid of a native and a heterologous signal peptide, and may specifically be heterologous or homologous to the host organism producing a POI. The function of the signal peptide is to allow the POI to be secreted to enter the endoplasmic reticulum. It is usually a short (3-60 amino acids long) peptide chain that directs the transport of a protein outside the plasma membrane, thereby making it easy to separate and purify a heterologous protein. Some signal peptides are cleaved from the protein by signal peptidase after the proteins are transported.

Exemplary signal peptides are signal sequences from S. cerevisiae alpha-mating factor prepro peptide and the signal peptides from the P. pastoris acid phosphatase gene (PHO1), the signal sequence of an oligosaccharyl transferase (OST1) and the extracellular protein X (EPX1) (WO2014067926A1) or chimeric fusions thereof.

Transformants as described herein can be obtained by introducing an expression vector DNA, e.g. plasmid DNA, into a host and selecting transformants which express a POI with high yields. Host cells are treated to enable them to incorporate foreign DNA by methods conventionally used for transformation of eukaryotic cells, such as the electric pulse method, the protoplast method, the lithium acetate method, and modified methods thereof. P. pastoris is preferably transformed by electroporation. Preferred methods of transformation for the uptake of the recombinant DNA fragment by the microorganism include chemical transformation, electroporation or transformation by protoplastation. Transformants described herein can be obtained by introducing such a vector DNA, e.g. plasmid DNA, into a host and selecting transformants which express the relevant protein or host cell metabolite with high yields.

A cell culture product can be produced by culturing the recombinant host cell line in an appropriate medium, isolating the expressed POI from the culture, and optionally purifying it by a suitable method.

Several different approaches for the production of the POI described herein are preferred. Substances may be expressed, processed and optionally secreted by transforming the yeast host cell with an expression vector harboring recombinant DNA encoding a relevant protein and at least one of the regulatory elements as described herein, preparing a culture of the transformed cell, growing the culture, inducing transcription and POI production, and recovering the product of the fermentation process.

The host cell described herein is specifically tested for its expression capacity or yield by the following test: ELISA, activity assay, HPLC, or other suitable tests.

It is understood that the methods disclosed herein may further include cultivating said recombinant host cells under conditions permitting the expression of the POI, either in the secreted form or else as intracellular product. A recombinant POI or a host cell metabolite can then be isolated from the cell culture medium and further purified by techniques well known to a person skilled in the art.

The term “cell culture” or “cultivation” (“culturing” is herein synonymously used), also termed “fermentation”, with respect to a host cell line is meant to be the maintenance of cells in an artificial, e.g., an in vitro environment, under conditions favoring growth, differentiation or continued viability, in an active or quiescent state, of the cells, specifically in a controlled bioreactor according to methods known in the industry. When cultivating, a cell culture is brought into contact with the cell culture media in a culture vessel or with substrate under conditions suitable to support cultivation of the cell culture. In certain embodiments, a culture medium as described herein is used to culture cells according to standard cell culture techniques that are well-known in the art for cultivating or growing yeast cells.

Cultivation of the yeast host cells may be in one or multiple phases.

According to a specific embodiment, the yeast cells are allowed to grow to a certain density in a first phase, before the carbonyl group is produced in a second or further phase. Cell density used for inoculating or starting the production phase may be OD600 of about 2 or more, specifically about 2.5, 3, 4, 5, 6 or more. The growth phase may be followed by an induction phase, wherein expression of the oxidase on the yeast cell surface is induced. The induction phase may also be included in the growth phase or the production phase.

According to another specific embodiment, cell growth and production of the carbonyl compound may be in a single phase. In this case, the medium used in the cultivation process comprises the respective substrate required for the production of the carbonyl compound from the beginning of the cultivation process.

Cell culture may be a batch process, or a fed-batch process. A batch process is a cultivation mode in which all the nutrients necessary for cultivation of the cells, and optionally including the substrates necessary for production of the carbonyl compounds described herein, are contained in the initial culture medium, without additional supply of further nutrients during fermentation. In a fed-batch process, a feeding phase takes place after the batch phase. In the feeding phase one or more nutrients, such as the substrate described herein, are supplied to the culture by feeding. In certain embodiments, the method described herein is a fed-batch process. Specifically, a host cell transformed with a nucleic acid construct encoding the fusion protein as described herein, is cultured in a growth phase medium and transitioned to an induction phase medium in order to produce the surface displayed oxidases described herein. Subsequently, the cells are transitioned to a reaction medium comprising the substrate described herein to produce a desired amount of the carbonyl compound described herein.

In another embodiment, host cells described herein are cultivated in continuous mode, e.g. a chemostat. A continuous fermentation process is characterized by a defined, constant and continuous rate of feeding of fresh culture medium into the bioreactor, whereby culture broth is at the same time removed from the bioreactor at the same defined, constant and continuous removal rate. By keeping culture medium, feeding rate and removal rate at the same constant level, the cultivation parameters and conditions in the bioreactor remain constant.

The POI produced according to a method described herein typically can be isolated and purified using state of the art techniques, including the increase of the concentration of the desired POI and/or the decrease of the concentration of at least one impurity.

Secretion of the recombinant expression products from the host cells is generally advantageous for reasons that include facilitating the purification process, since the products are recovered from the culture supernatant rather than from the complex mixture of proteins that results when yeast cells are disrupted to release intracellular proteins.

The cultured transformant cells may also be ruptured sonically or mechanically, enzymatically or chemically to obtain a cell extract containing the desired POI, from which the POI is isolated and purified.

As isolation and purification methods for obtaining a recombinant polypeptide or protein product, methods, such as methods utilizing difference in solubility, such as salting out and solvent precipitation, methods utilizing difference in molecular weight, such as ultrafiltration and gel electrophoresis, methods utilizing difference in electric charge, such as ion-exchange chromatography, methods utilizing specific affinity, such as affinity chromatography, methods utilizing difference in hydrophobicity, such as reverse phase high performance liquid chromatography, and methods utilizing difference in isoelectric point, such as isoelectric focusing may be used.

As isolation and purification methods the following standard methods are preferred: Cell disruption (if the POI is obtained intracellularly), cell (debris) separation and wash by Microfiltration or Tangential Flow Filter (TFF) or centrifugation, POI purification by precipitation or heat treatment, POI activation by enzymatic digest, POI purification by chromatography, such as ion exchange (IEX), hydrophobic interaction chromatography (HIC), Affinity chromatography, size exclusion (SEC) or HPLC Chromatography, POI precipitation of concentration and washing by ultrafiltration steps.

The isolated and purified POI or metabolite can be identified by conventional methods such as Western blot, HPLC, activity assay, or ELISA.

If the POI is a protein homologous to the host cell, i.e. a protein which is naturally occurring in the host cell, the expression of the POI in the host cell may be modulated by the exchange of its native promoter sequence with a heterologous promoter sequence.

According to a specific embodiment, the POI production method employs a recombinant nucleotide sequence encoding the POI, which is provided on a plasmid suitable for integration into the genome of the host cell, in a single copy or in multiple copies per cell. Integration into the genome, specifically the chromosomes, of the host cell, may for example be achieved using homologous recombination as described herein. However, any suitable method for integration of the recombinant nucleic acid sequence into the host genome may be used.

The recombinant nucleotide sequence encoding the POI may also be provided on an autonomously replicating plasmid in a single copy or in multiple copies per cell. The recombinant nucleotide sequence encoding the POI may also be provided in an expression cassette on an artificial chromosome, in a single copy or in multiple copies per cell.

The preferred method as described herein employs a plasmid, which is a eukaryotic expression vector, preferably a yeast expression vector. Expression vectors may include but are not limited to cloning vectors, modified cloning vectors and specifically designed plasmids. A preferred expression vector as used in a method described herein may be any expression vector suitable for expression of a recombinant gene in a host cell and is selected depending on the host organism. The recombinant expression vector may be any vector which is capable of replicating in or integrating into the genome of the host organisms, also called host vector, such as a yeast vector, which carries a DNA construct as described herein.

Specifically, plasmids derived from pPICZ, pGAPZ, pPIC9, pPICZalpha, pGAPZalpha, pPIC9K, pGAPHis, pPUZZLE, are used as a vector. Specifically, plasmids derived from pPpT4 (Näätsaari et al. 2012) or pJ series vectors (commercially available from Biogrammatics Inc.) are used as a vector.

According to a preferred embodiment, a recombinant construct is obtained by ligating the relevant genes into a vector. These genes can be stably integrated into the host cell genome by transforming the host cell using such vectors. The polypeptides encoded by the genes can be produced using the recombinant host cell line by culturing a transformant, thus obtained in an appropriate medium, isolating the expressed POI from the culture, and purifying it by a method appropriate for the expressed product, in particular to separate the POI from contaminating proteins.

Expression vectors may comprise one or more phenotypic selectable markers, e.g. a gene encoding a protein that confers antibiotic resistance or that supplies an autotrophic requirement. Yeast vectors commonly contain an origin of replication from a yeast plasmid, an autonomously replicating sequence (ARS), a centromere (CEN) sequence or alternatively, a sequence used for integration into the host genome, a promoter region, sequences for polyadenylation, sequences for transcription termination, and a selectable marker.

The procedures used to ligate the DNA sequences, regulatory elements and the gene(s) coding for the POI, the promoter and the terminator, respectively, and to insert them into suitable vectors containing the information necessary for integration or host replication, are well-known to persons skilled in the art, e.g. described by J. Sambrook et al., (A Laboratory Manual, Cold Spring Harbor, 1989).

The DNA construct as provided to obtain a recombinant host cell may be prepared synthetically by established standard methods, e.g. the phosphoramidite method. The DNA construct may also be of genomic or cDNA origin, for instance obtained by preparing a genomic or cDNA library and screening for DNA sequences coding for all or part of the polypeptide by hybridization using synthetic oligonucleotide probes in accordance with standard techniques (Sambrook et al., Molecular Cloning: A Laboratory Manual, Cold Spring Harbor, 1989). Finally, the DNA construct may be of mixed synthetic and genomic, mixed synthetic and cDNA or mixed genomic and cDNA origin prepared by annealing fragments of synthetic, genomic or cDNA origin, as appropriate, the fragments corresponding to various parts of the entire DNA construct, in accordance with standard techniques.

The term “sequence identity” as used herein is understood as the relatedness between two amino acid sequences or between two nucleotide sequences and described by the degree of sequence identity or sequence complementarity. The sequence identity of a variant, homologue or orthologue as compared to a parent nucleotide or amino acid sequence indicates the degree of identity of two or more sequences. Two or more amino acid sequences may have the same or conserved amino acid residues at a corresponding position, to a certain degree, up to 100%. Two or more nucleotide sequences may have the same or conserved base pairs at a corresponding position, to a certain degree, up to 100%.

Sequence similarity searching is an effective and reliable strategy for identifying homologs with excess (e.g., at least 50%) sequence identity. Sequence similarity search tools frequently used are e.g., BLAST, FASTA, and HMMER.

Sequence similarity searches can identify such homologous proteins or polynucleotides by detecting excess similarity, and statistically significant similarity that reflects common ancestry. Homologues may encompass orthologues, which are herein understood as the same protein in different organisms, e.g., variants of such protein in different organisms or species.

To determine the % complementarity of two complementary sequences, one of the two sequences needs to be converted to its complementary sequence before the % complementarity can then be calculated as the % identity between the first sequence and the second converted sequences using the above-mentioned algorithm.

“Percent (%) identity” with respect to an amino acid sequence, homologs and orthologues described herein is defined as the percentage of amino acid residues in a candidate sequence that are identical with the amino acid residues in the specific polypeptide sequence, after aligning the sequence and introducing gaps, if necessary, to achieve the maximum percent sequence identity, and not considering any conservative substitutions as part of the sequence identity. Those skilled in the art can determine appropriate parameters for measuring alignment, including any algorithms needed to achieve maximal alignment over the full length of the sequences being compared. In case of percentages determined for sequence identities, it is possible that arithmetical decimal places may result which are not possible with regard to full nucleotides or amino acids. In this case, the percentages shall be rounded up to whole nucleotides or amino acids.

For purposes described herein, the sequence identity between two amino acid sequences is determined using the NCBI BLAST program version 2.2.29 (Jan.-06-2014) with blastp set at the following exemplary parameters: Program: blastp, Word size: 6, Expect value: 10, Hitlist size: 100, Gapcosts: 11.1, Matrix: BLOSUM62, Filter string: F, Genetic Code: 1, Window Size: 40, Threshold: 21, Composition-based stats: 2.

“Percent (%) identity” with respect to a nucleotide sequence e.g., of a nucleic acid molecule or a part thereof, in particular a coding DNA sequence, is defined as the percentage of nucleotides in a candidate DNA sequence that is identical with the nucleotides in the DNA sequence, after aligning the sequence and introducing gaps, if necessary, to achieve the maximum percent sequence identity, and not considering any conservative substitutions as part of the sequence identity. Alignment for purposes of determining percent nucleotide sequence identity can be achieved in various ways that are within the skill in the art, for instance, using publicly available computer software. Those skilled in the art can determine appropriate parameters for measuring alignment, including any algorithms needed to achieve maximal alignment over the full length of the sequences being compared.

Optimal alignment may be determined with the use of any suitable algorithm for aligning sequences, non-limiting examples of which include the Smith-Waterman algorithm, the Needleman-Wunsch algorithm, algorithms based on the Burrows-Wheeler Transform (e.g., the Burrows Wheeler Aligner), ClustalW, Clustal X, BLAT, Novoalign (Novocraft Technologies; available at novocraft.com), ELAND (Illumina, San Diego, CA), SOAP (available at soap.genomies.org.cn), and Maq (available at maq.sourceforge.net).

### EXAMPLES

The Examples which follow are set forth to aid in the understanding of the invention but are not intended to, and should not be construed to limit the scope of the invention in any way. The Examples do not include detailed descriptions of conventional methods, e.g., cloning, transfection, and basic aspects of methods for overexpressing proteins in microbial host cells. Such methods are well known to those of ordinary skill in the art.

The goal of this project was to enable a quick and reliable generation of new Pichia pastoris strains with improved intracellular or secreted protein production capacities, thereby avoiding currently applied unpredictable and time consuming random approaches which are used to increase the efficiency of protein production above usual levels which are obtained by classical cloning and transformation techniques such as targeted integration of defined expression cassettes consisting of a gene of interest linked to a promoter and transcription terminator and a selection marker cassette into the genome of the host cell Komagataella phaffii (Pichia pastoris ). The main concept was to search for extraordinary efficient expression clones for any target which was expressed in previous work, to study surprising highly producing phenotypes of clones (Jackpot clones) by next generation sequencing (NGS) and data analysis using a bioinformatics approach and to evaluate the reproducibility of effects or to make use of the identified genome changes in other ways in order to produce other target proteins than the original protein, which is produced by the original Jackpot clone. Therefore, it was a goal of this invention to evaluate possibilities for a more rational, reliable and time efficient approach for the generation of efficient protein producers. Core of the performed studies was the evaluation of a systematic transfer of the beneficial features of jackpot host strain backgrounds to alternative additional protein production targets.

Application of new platform strains will accelerate the development process and reduce the development costs for competitive industrial protein production. Jackpot strains will replace currently used standard hosts cells for industrial gene expression.

10 Jackpot strains with unexpectedly high levels of recombinant protein were selected from the bisy strain collection and sequenced by Illumina sequencing. The Illumina genome re-sequencing data of the selected strains were analyzed focusing on the copy number and integration locus of the expression cassette. 6 strains were identified to be single copy, which makes them especially interesting for the development of new platform strains as an alternative to the commonly used approach of multi copy expression strain, which are frequently more productive than transformants containing single copies of expression cassettes integrated in their genome.

To analyze whether these strains can be used for the efficient production of a specific recombinant protein but also other proteins of interest, the original recombinant POI was removed and replaced by an empty cassette. In a second step the original POI or another protein of interest was re-inserted into the genome of the yeast cell, either target-specific at the specific locus by integrating into the empty expression cassette or replacing the initial expression cassette, or randomly.

In either case, the disruption of the endogenous gene at the integration site of the original POI is maintained, which makes disruption of the genes at the specific locus the reason for the successful expression of different recombinant proteins by one strain.

Results showed that Jackpot strains can be used for the generation of new platform strains, on a technical level (genotype) and also on a practical level for enhanced protein production (phenotype).

### Example 1—Materials & Methods

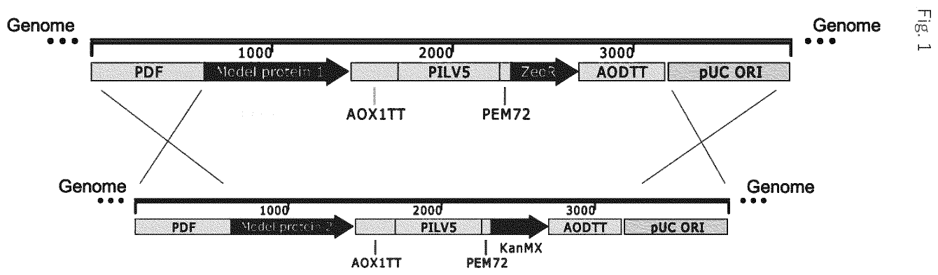

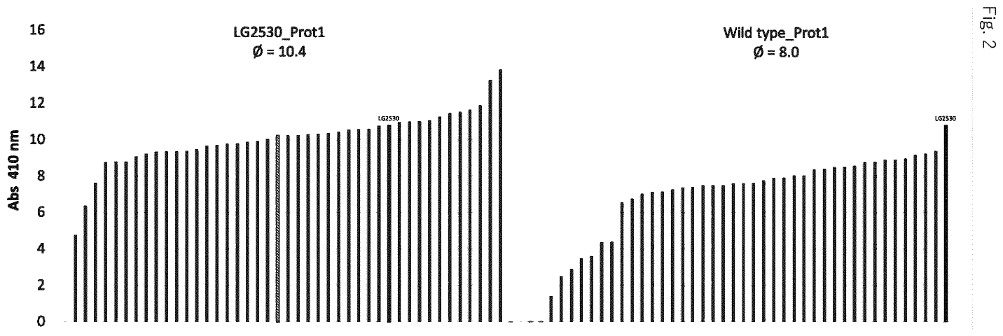

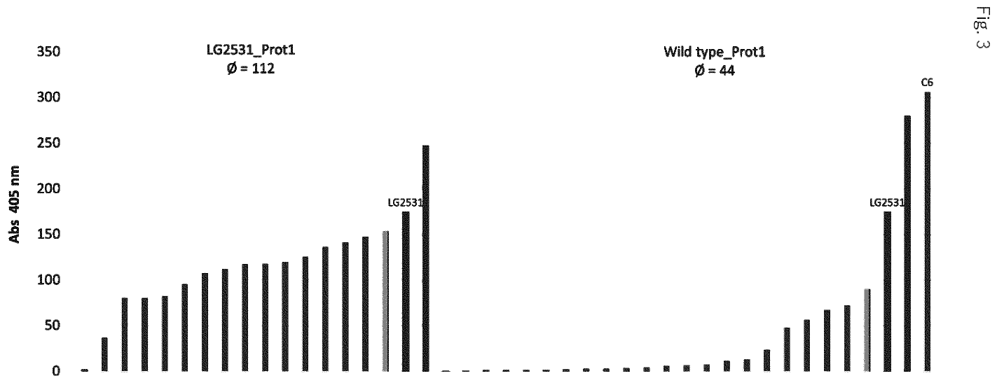

To evaluate the possibility of a transfer of the identified genetic changes to other industrially relevant model proteins, but also to verify the effect for the specific POI a landing pad strategy based on homologous recombination was used (FIG. 1). To exchange the gene of interest of the Jackpot strains, an expression cassette with the same 5′ (PDF promoter) and 3′ (pUC origin) genetic elements, but another antibiotic marker, was used. Empty platform strains were generated using the same strategy, however the model protein was replaced by a stuffer fragment.

Jackpot strains were transformed with an empty cassette and subsequently re-transformed with model protein 1 (original protein of interest) and/or model protein 2 (another protein of interest), allowing for random as well as locus specific integration. In parallel the wild type strain, either BSYBG10 or BSYBG11, was transformed.

The expression cassette was derived from Smil linearized plasmid pBSY5S1Z which is based on the vector pPpT4 described by Näätsaari et al. (PLOS One 7 (2012). For gene expression the orthologous promoter, PDF, was used, which is inducible by derepression (described in WO2017109082A1). The promoter and POI were cloned seamlessly (i.e., without any restriction enzyme cleavage sites or linker sequences between the promoter and the start codon) using Gibson assembly and 40 bp of homologous regions. Markers were used as described by Näätsaari et al. (PLoS One 7 (2012): e39720).

For cultivation, a fast, reliable and easy-to-do protocol for high-throughput screening in 96-DWPs was used as published by Weis et al. FEMS Yeast Research, 2004, 5, 179-189.

About 50 clones were cultivated per strain in small scale using glycerol as sole carbon source. For methanol free-cultivation following media were used. BMG1 (buffered minimal dextrose containing 1% glycerol: 1.34% yeast nitrogen base w/o amino acids, 4×10−5% biotin, 200 mM potassium phosphate buffer, pH 6.0 and 1% glucose). BMG0.5 (buffered minimal methanol containing 0.5% glycerol: 1.34% yeast nitrogen base w/o amino acids, 4×10−5% biotin, 200 mM potassium phosphate buffer, pH 6.0 and 0.5% glycerol). BMG2.5 (buffered minimal methanol containing 2.5% glycerol: 1.34% yeast nitrogen base w/o amino acids, 4×10−5% biotin, 200 mM potassium phosphate buffer, pH 6.0 and 2.5% glycerol). After 60 hours of growth in 250 μL of BMG1, the cultures were induced by addition of 250 μL BMG0.5, followed by 3× addition of 50 μL BMG2.5 every 12 h. 12 hours after the last induction, cells were harvested at 4000 rpm and supernatants were evaluated for enzyme activity or total secreted protein.

TABLE 1
Integration sites of original POI in selected strains, numbering
according to the genome sequence published by Sturmberger
et al., 2016, Details of strains listed below.

| Strain | Parental | | | |
|---|---|---|---|---|
| Number# | Strain | Plasmid | CN | Integration site |
| LG2530 | BSYBG10 | pBSYPDFsec_blunt_{—} | single | LT962477.1: 65654-65655 |
| | | appA_Mut_V3_6D | | |
| LG2531 | BSYBG11 | pBSY5S1Z_{—} | single | LT962476.1: 949930-949931 |
| | | POXsyn17corr_4A | | |
| LG2532** | BSYBG11 | pBSY5S1Z_{—} | single | LT962479.1: 1303485-1303486 |
| | | POXsyn17_C6 | | |

### Example 2—Novel Strain LG2530

Integration of the original GOI (encoding Model Protein 1) at the integration site listed in Table 1 affected the following genes in Strain LG2530 (numbering according to Valli et al., 2016):
ALG9 (64999 . . . 66840): Mannosyltransferase, involved in N-linked glycosylation; catalyzes both the transfer of seventh mannose residue on B-arm and ninth mannose residue on the C-arm from Dol-P-Man to lipid-linked oligosaccharides; mutation of the human ortholog causes type 1 congenital disorders of glycosylation

The strain also comprises an indel, which is only present in this strain: LT962479 position 558627 due to GC->G causes a frameshift in the following genes:
ACIB2EUKG772361 (complement (556,182 . . . 558,806)): “CCR4-NOT complex (Transcriptional regulatory complex involved in mRNA initiation, elongation, and degradation) subunit”, and
PP7,435_CHR4-0335 (complement (556182 . . . 558806)): Protein of unknown function; in S. cerevisiae the green fluorescent protein (GFP)-fusion protein localizes to the cell periphery, cytoplasm, bud, and bud neck; potential Cdc28p substrate; similar to Skg4p; relocalizes from bud neck to cytoplasm upon DNA replication stress; Pichia pastoris does not have the paralog CAF120.

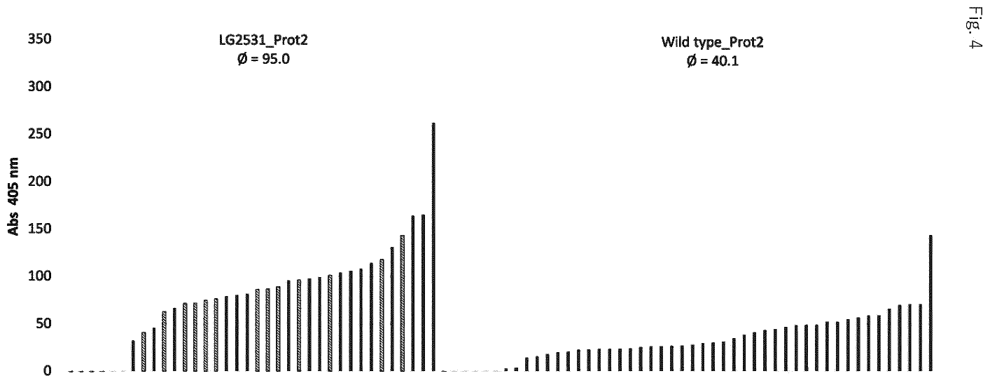

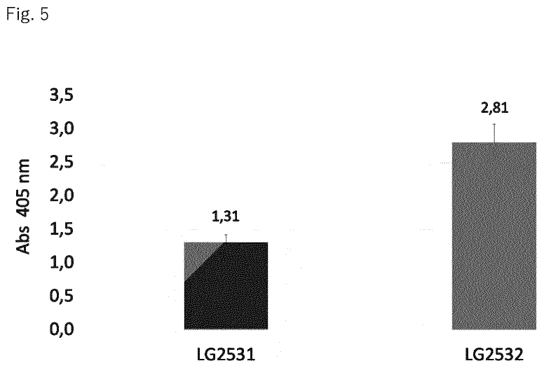

Confirming the phenotype of strain LG2530, the average activity of LG2530 clones expressing model protein 1 was found 25% higher than the activity of wild type clones (FIG. 2). Further, the activity level of LG2530 transformants was comparable to the activity of the unmodified parental Jackpot strain, confirming the validity of the experiment.

### Example 3—Novel Strain LG2531

Integration of the original GOI (encoding Model Protein 1) at the integration site listed in Table 1 affected the following genes in Strain LG2531 (numbering according to Valli et al., 2016):
SRB8 (complement (945553 . . . 950169)): Subunit of the RNA polymerase Il mediator complex; associates with core polymerase subunits to form the RNA polymerase II holoenzyme; essential in S. cerevisiae for transcriptional regulation; involved in glucose repression.

When re-integrating model protein 1 into strain LG2531, a lot more clones with higher activity were found in comparison to the experiment in which the wild type strain was used (average relative absorption of 112 compared to 44, FIG. 3), confirming the super-secreter phenotype of this strain. Interestingly, the majority of the clones was again found to have the cassette integrated in the LG2531 locus. While many wild type transformants were found to have very low activity, again one new Jackpot clone with putative super-secreter phenotype was identified, i.e. C6 (from here on referred to as LG2532).

As for model protein 1, also activities of LG2531 clones expressing model protein 2 were found significantly increased compared to the respective wild type clones. While the LG2531 clones reached an average relative absorption level of 95, the wild type clones reached a level of only 40 (FIG. 4).

### Example 4—Novel Strain LG2532

Integration of the original GOI (encoding Model Protein 1) at the integration site listed in Table 1 affected the following genes in Strain LG2532 (numbering according to Valli et al., 2016):
ACIB2EUKG772803 (1301453-1303573): Ferric reductase, reduces siderophore-bound iron prior to uptake by transporters

The newly discovered strain LG2532 was benchmarked in a rescreening against Jackpot strain LG2531. Strain LG2532 showed significantly enhanced activity when studied on glycerol, i.e. 2-fold improvement compared to strain LG2531 (FIG. 5).

### Example 5—Expression of Various Peroxygenases and Peroxidases in the Novel Strain LG2531

The K. phaffii chassis LG2531 (having a frame disruption of the SRB8 gene, see SEQ ID NO:19 and Example 3) was used as a host for expression of 13 different putative unspecific peroxygenases (UPOs). To evaluate the effect of the host strain on peroxygenase/peroxidase expression the same enzymes were also expressed in the wildtype strain BSYBG11.

For the recombinant expression of the different UPOs the PDF promoter (P_HpFMD) and the alpha-mating factor signal peptide from S. cerevisiae (MATalphaD) or the native signal peptide was used. The mean ABTS activity in the cultivation supernatant of multiple transformants was used for comparison of the two strains.

As can be seen in Table 2 enzyme expression in the modified chassis (Jackpot (JP) chassis) was enhanced in comparison to the wildtype strain. A positive effect can be seen regardless of the organism of origin, type of UPO (group I or II, short or long) or the signal peptide used for secretion.

TABLE 2
Activities of peroxygenases/peroxidases (SEQ ID Nos 1-13) expressed in the JP chassis
LG2531 and the wildtype strain BSYBG11. Expression was evaluated by determining ABTS
activity in the cultivation supernatant of the respective expression strains. Additionally,
effect of the JP chassis, classification, protein ID and organism of origin of the
expressed UPOs, as well as signal peptide used for secretion is given.

| Ø Activity | | | | | | |
|---|---|---|---|---|---|---|
| ABTS | Ø Activity | | UPO | | | |
| [U/L] JP | ABTS [U/L] | | Classification | | Organism of | Secretion |
| chassis | WT strain | Effect | [Group I or II] | Protein ID | origin | signal |
| 73.1 | 42.3 | ++ | I | CDP25756.1 | P. anisera | nat. |
| 2.2 | 1.3 | ++ | I | OTB17553.1 | Daldinia sp. | nat. |
| 5.2 | 2.0 | ++ | II | TFK24496.1 | C. marcescibilis | nat. |
| 6.9 | 5.8 | + | I | KAB8223135.1 | A. novoparasiticus | nat. |
| 2.2 | 1.7 | + | I | OTB09996.1 | Daldinia sp. | α |
| 12.3 | 7.0 | ++ | I | XP_020060613.1 | A. aculeatu | α |
| 6.5 | 4.8 | + | II | RXW15716.1 | P. aberdarensis | nat. |
| 12.2 | 8.4 | + | I | OJJ73116.1 | A. brasiliensis | α |
| 4.7 | 1.6 | ++ | I | PPR06026.1 | G. dilepis | nat. |
| 0.17 | <0.1 | ++ | II | KIK53163.1 | G. luxurians | nat. |
| 8.8 | 6.8 | + | I | OTA57433.1 | Hypoxylon sp. | α |
| 3.9 | 2.5 | + | I | XP_001390900.2 | A. niger | nat. |

### Example 6—Testing Effect of Promoter

Since the positive effect of the JP chassis on UPO expression cannot be attributed to the secretion signal or UPO specific properties (origin organism, type of UPO), the promoter controlling expression of the GOI (gene of interest) was further investigated.

The UPO of Hypoxylon sp. (OTA57433.1) was expressed under the control of three different promoter sequences, the PDF (P_HpFMD), which has been used previously and the PAOX1 and the P_{GAP} which are the state of the art inducible and constitutive promoter for K. phaffii , respectively. All constructs were expressed in the JP chassis as well as the wildtype BYSBG11.

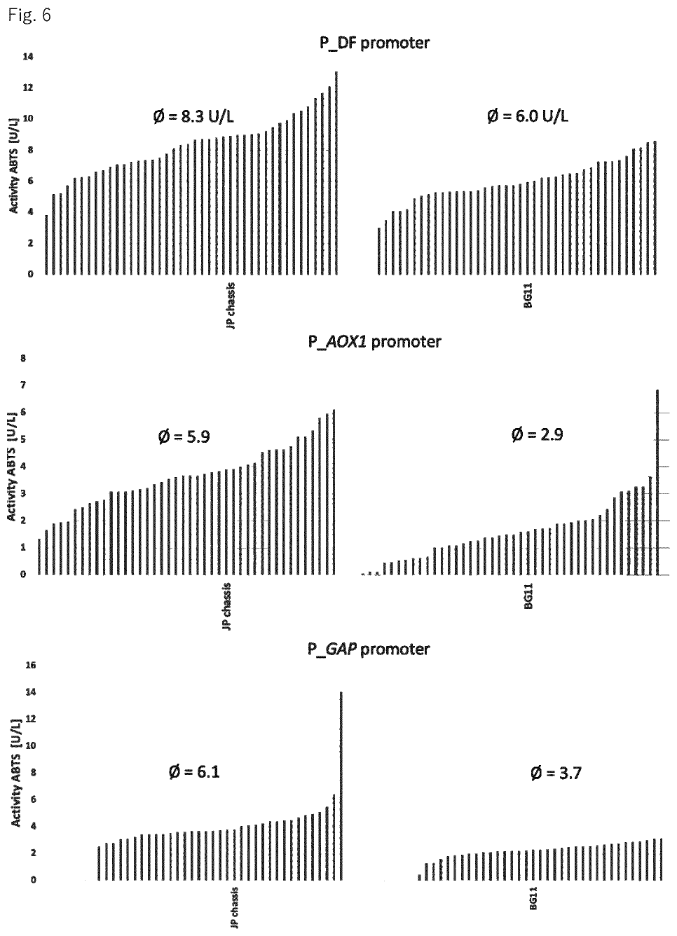

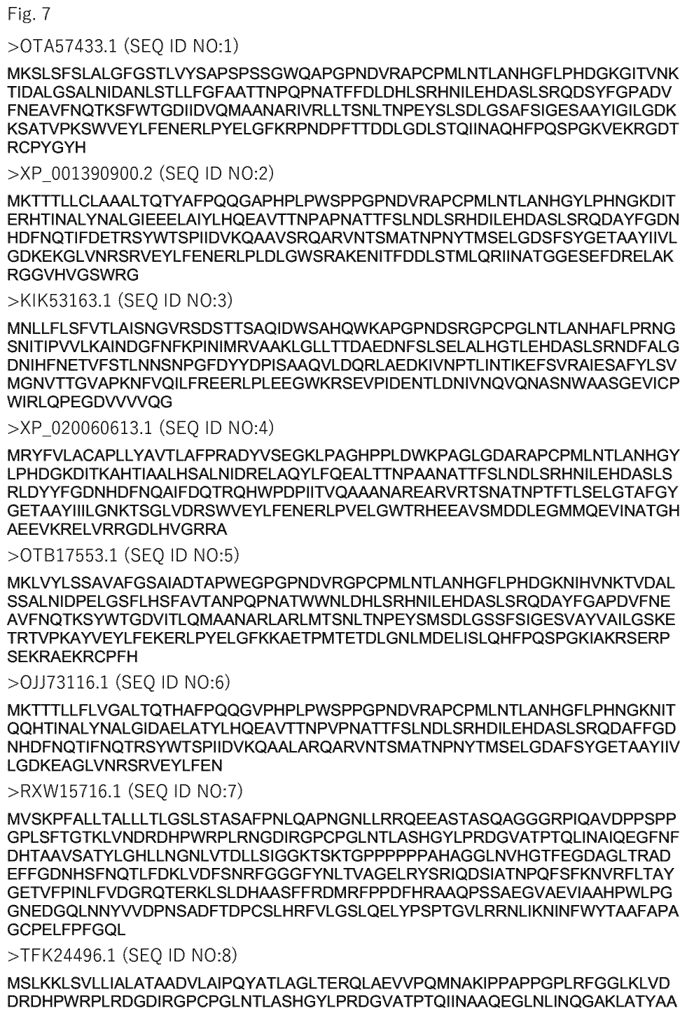

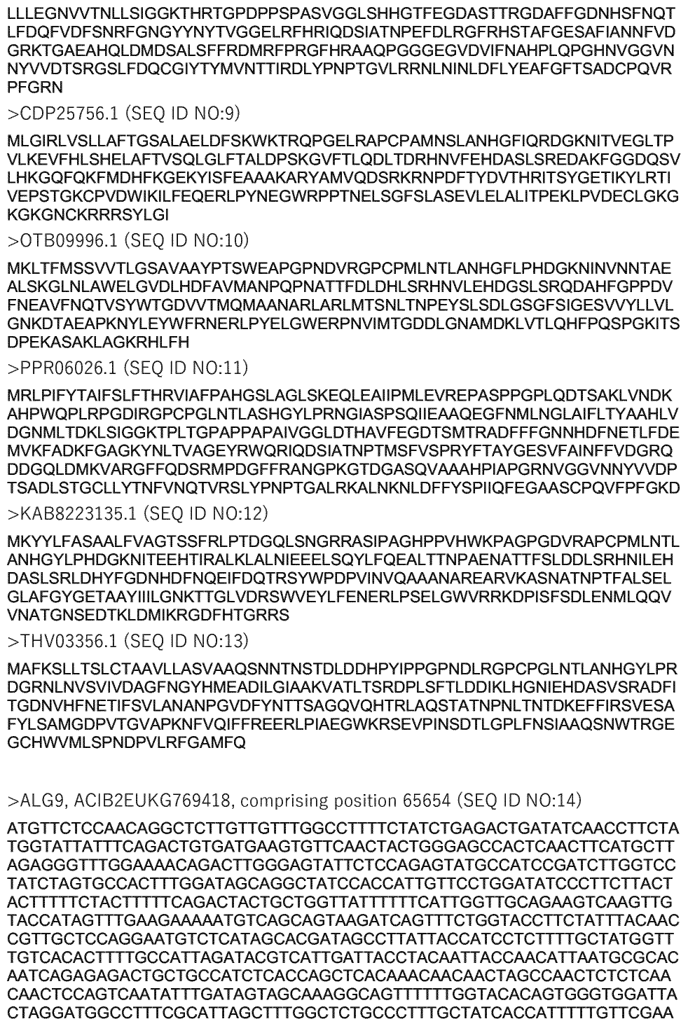

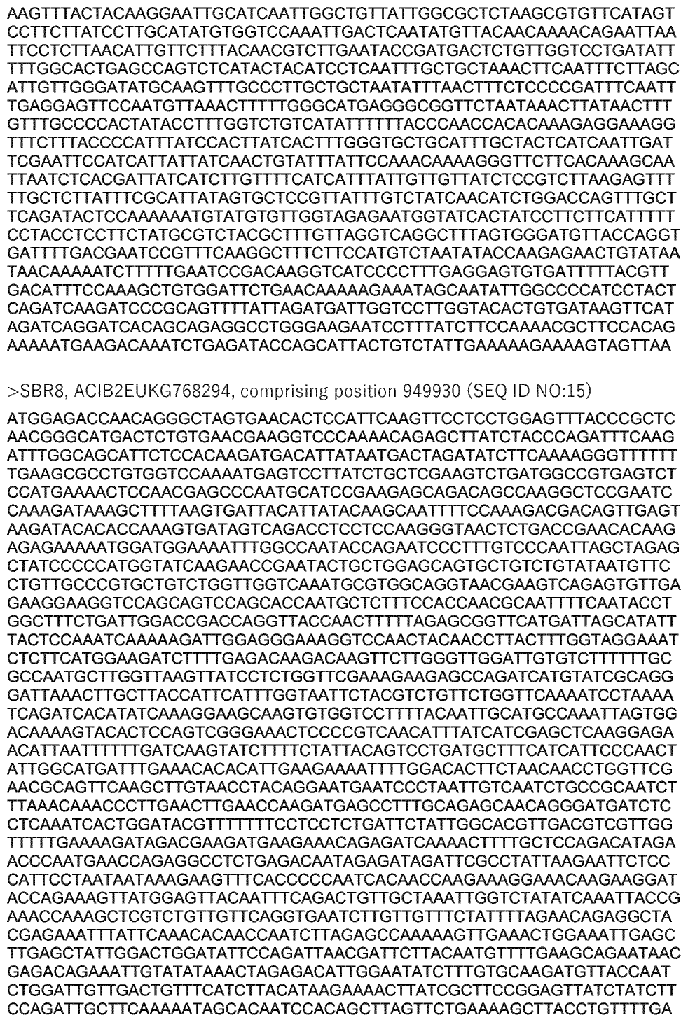

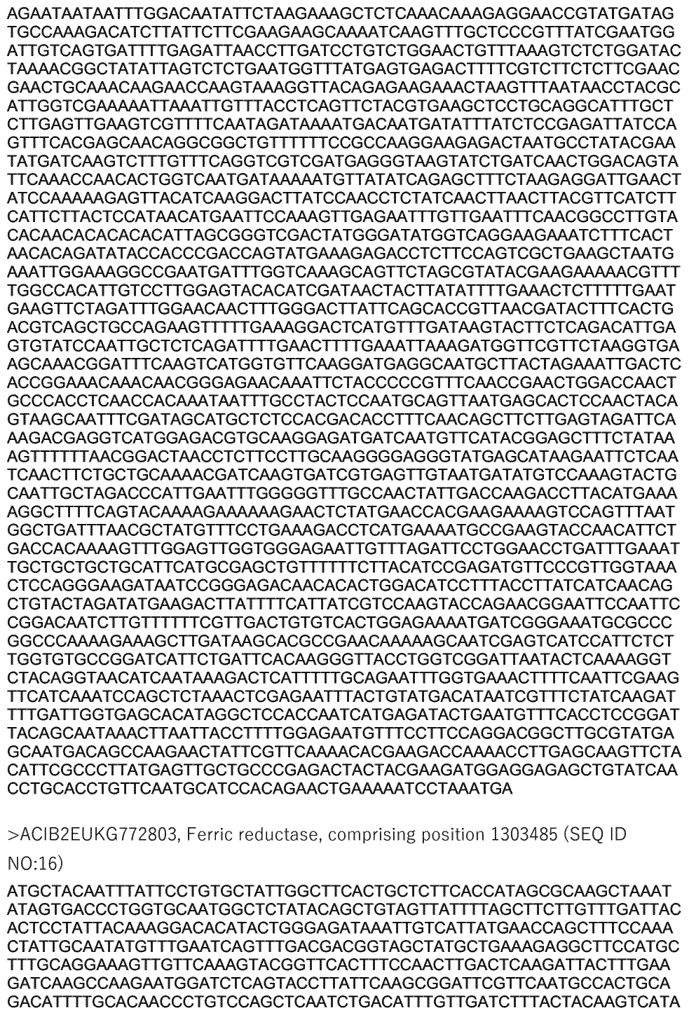

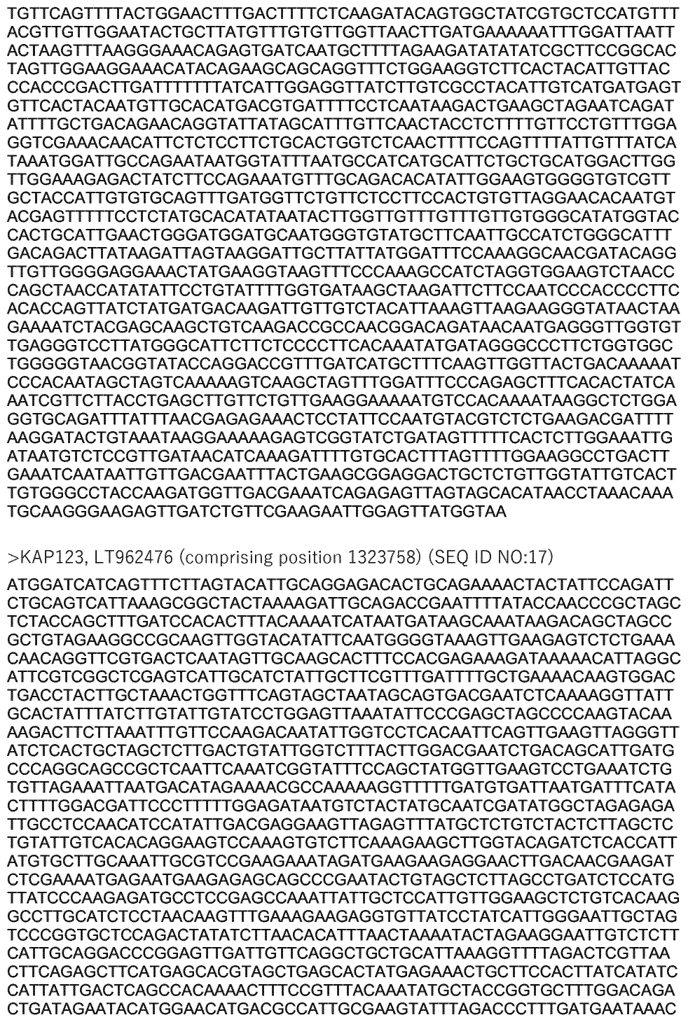

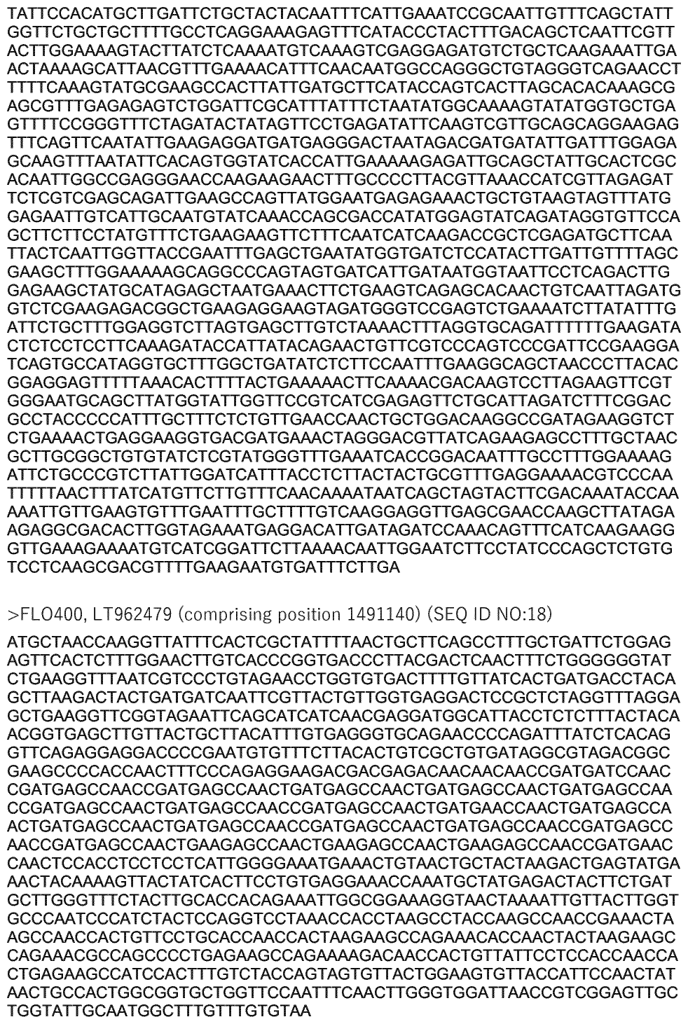

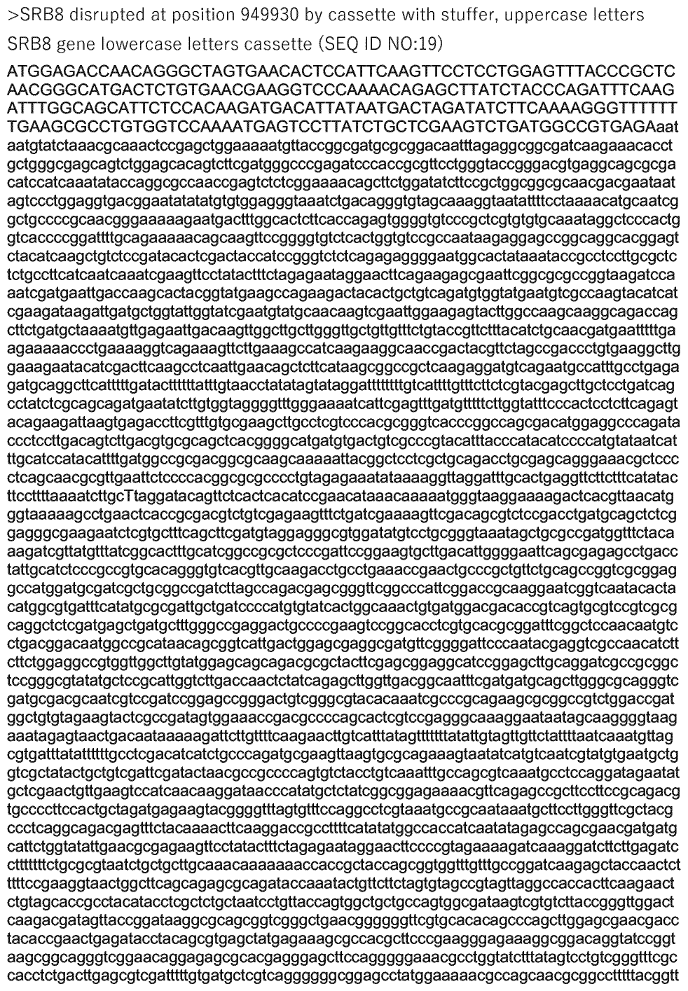

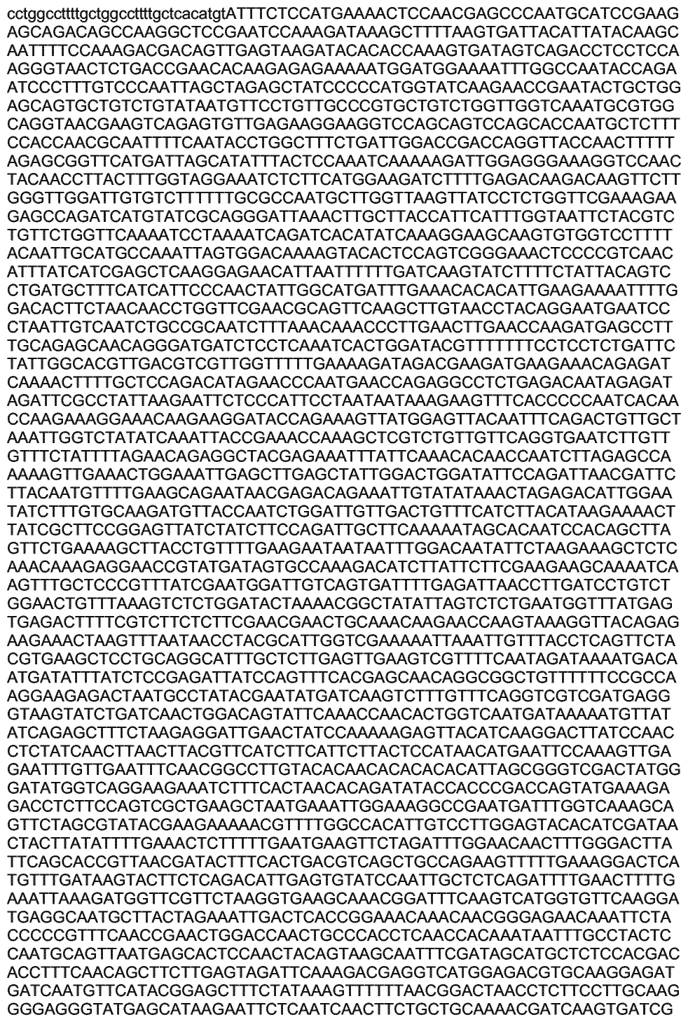

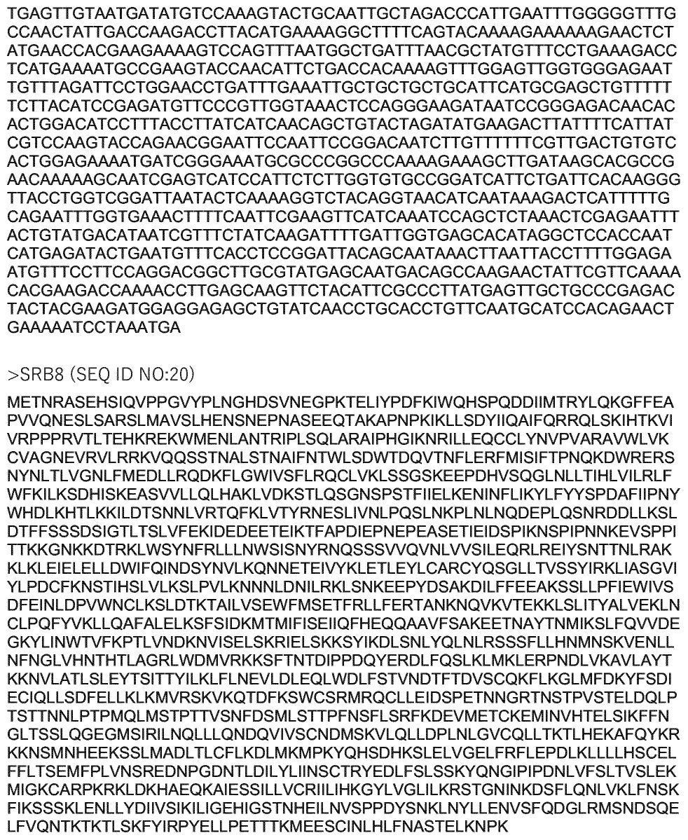

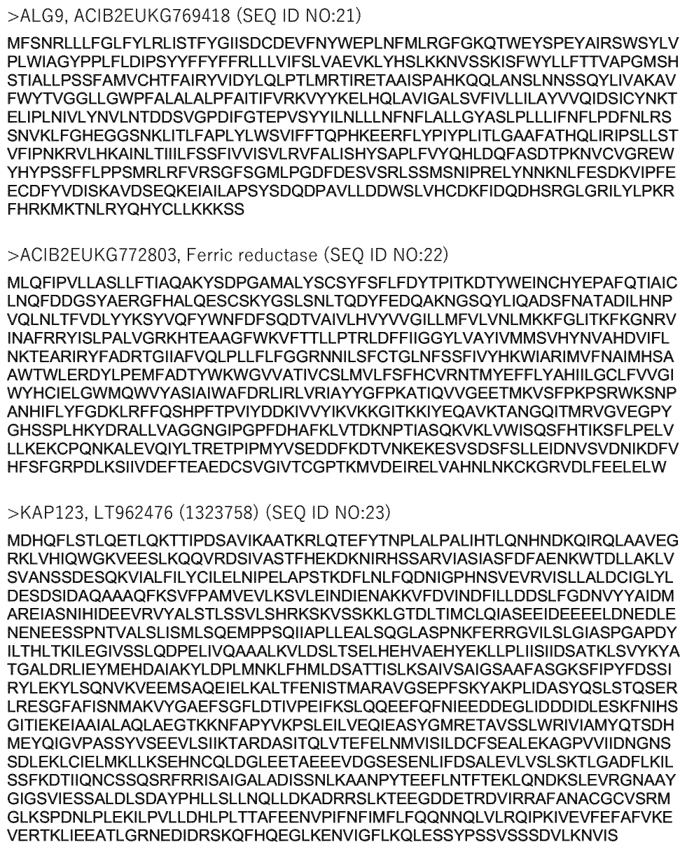

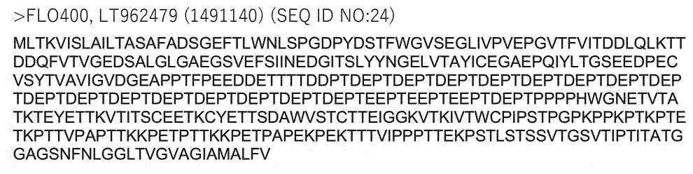

As can be seen in FIG. 6 the JP chassis is superior for the expression of the UPOs, irrespective of the used promoter sequence.

### REFERENCES

Aw R, Polizzi KM. 2013. Can too many copies spoil the broth? Microb Cell Fact 12:128. doi: 10.1186/1475-2859-12-128.
Brooks, C.L., Morrison, M., Lemieux, M.J., 2013. Rapid expression screening of eukaryotic membrane proteins in Pichia pastoris . Protein Sci. 22, 425-433. https://doi.org/10.1002/pro.2223
Cregg, J.M., Barringer, K.J., Hessler, A.Y., Madden, K.R., 1985. Pichia pastoris as a host system for transformations. Mol. Cell. Biol. 5, 3376-85.
Cregg, J.M., Cereghino, J.L., Shi, J., Higgins, D.R., 2000. Recombinant protein expression in Pichia pastoris . Mol. Biotechnol. 16, 23-52. https://doi.org/10.1385/MB:16:1:23
Fischer, J.E., Hatzl, A., Weninger, A., Schmid, C. Glieder, A., 2019. Methanol Independent Expression by Pichia pastoris Employing De-repression Technologies, J Vis Exp., 143. doi: 10.3791/58589
Gasser, B., Mattanovich, D., Buchetics, M., 2014. Recombinant host cell for expressing proteins of interest, International Patent Application.
Larsen, S., Weaver, J., de Sa Campos, K., Bulahan, R., Nguyen, J., Grove, H., Huang, A., Low, L., Tran, N., Gomez, S., Yau, J., Ilustrisimo, T., Kawilarang, J., Lau, J., Tranphung, M., Chen, I., Tran, C., Fox, M., Lin-Cereghino, J., Lin-Cereghino, G.P., 2013. Mutant strains of Pichia pastoris with enhanced secretion of recombinant proteins. Biotechnol. Lett. 35, 1925-1935. https://doi.org/10.1007/s10529-013-1290-7
Liang S, Zou C, Lin Y, Zhang X, Ye Y. Identification and characterization of P GCW14: a novel, strong constitutive promoter of Pichia pastoris . Biotechnol Lett. 2013 Nov;35(11):1865-71. doi: 10.1007/s10529-013-1265-8. Epub 2013 Jun 26. PMID: 23801118.
Lin-cereghino, J., Hashimoto, M.D., Moy, A., Castelo, J., Orazem, C.C., Kuo, P., Xiong, S., Gandhi, V., Hatae, C.T., Chan, A., Lin-cereghino, G.P., 2008. Direct selection of Pichia pastoris expression strains using new G418 resistance vectors 293-299. https://doi.org/10.1002/yea
Lin-Cereghino, J., Lin-Cereghino, G.P., 2007. Vectors and strains for expression. Methods Mol. Biol. 389, 11-26. https://doi.org/10.1007/978-1-59745-456-8_2
Näätsaari, L., Mistlberger, B., Ruth, C., Hajek, T., Hartner, F.S., Glieder, A., 2012. Deletion of the Pichia pastoris ku70 homologue facilitates platform strain generation for gene expression and synthetic biology. PLoS One 7. https://doi.org/10.1371/journal.pone.0039720
Naranjo, C.A., Jivan, A.D., Vo, M.N., De, K.H., Campos, S., Deyarmin, J.S., Hekman, R.M., Uribe, C., Hang, A., Her, K., Fong, M.M., Choi, J.J., Chou, C., Rabara, T.R., Myers, G., Moua, P., Thor, D., Risser, D.D., Vierra, C.A., Franz, A.H., Lin-Cereghino, J., Lin-Cereghino, G.P., 2019. Role of BGS13 in the Secretory Mechanism of Pichia pastoris . https://doi.org/10.1128/AEM.01615-19
Qin X, Qian J, Yao G, Zhuang Y, Zhang S, Chu J., 2011. GAP promoter library for fine-tuning of gene expression in Pichia pastoris . Appl Environ Microbiol. 77(11):3600-8. doi: 10.1128/AEM.02843-10. Epub 2011 Apr 15.PMID: 21498769
Schwarzhans, J.-P., Wibberg, D., Winkler, A., Luttermann, T., Kalinowski, J., Friehs, K., 2016. Integration event induced changes in recombinant protein productivity in Pichia pastoris discovered by whole genome sequencing and derived vector optimization. Microb. Cell Fact. 15, 84. https://doi.org/10.1186/s12934-016-0486-7
Sturmberger et al. 2016. Refined Pichia pastoris reference genome sequence. Journal of Biotechnology. 235:121-131. https://doi.org/10.1016/j.jbiotec.2016.04.023
Sunga, A.J., Tolstorukov, I., Cregg, J.M., 2008. Posttransformational vector amplification in the yeast Pichia pastoris . FEMS Yeast Res. 8, 870-6. https://doi.org/10.1111/j.1567-1364.2008.00410.x
Valli et al., 2016. Curation of the genome annotation of Pichia pastoris (Komagataella phaffii) CBS7435 from gene level to protein function. FEMS Yeast Research. 16(6). https://doi.org/10.1093/femsyr/fow051
Vogl T., Glieder A.N., 2013. Regulation of Pichia pastoris promoters and its consequences for protein production. Biotechnol. 30(4):385-404. doi: 10.1016/j.nbt.2012.11.010. Epub 2012 Nov 16.
Vogl T., Sturmberger L, Kickenweiz T, Wasmayer R, Schmid C, Hatzl AM, Gerstmann MA, Pitzer J, Wagner M, Thallinger GG, Geier M, Glieder A., 2016. A Toolbox of Diverse Promoters Related to Methanol Utilization: Functionally Verified Parts for Heterologous Pathway Expression in Pichia pastoris. ACS Synth Biol. 5(2):172-86. doi: 10.1021/acssynbio.5b00199. Epub 2015 Dec 11.PMID: 26592304
Vogl, T., Gebbie, L., Palfreyman, R.W., Speight, R., 2018a. Effect of Plasmid Design and Type of Integration Event on Recombinant Protein Expression in Pichia pastoris. Appl. Environ. Microbiol. 84, e02712-17.
Vogl, T., Kickenweiz, T., Pitzer, J., Sturmberger, L., Weninger, A., Biggs, B.W., Köhler, E.-M., Baumschlager, A., Fischer, J.E., Hyden, P., Wagner, M., Baumann, M., Borth, N., Geier, M., Ajikumar, P.K., Glieder, A., 2018b. Engineered bidirectional promoters enable rapid multi-gene co-expression optimization. Nat. Commun. 9, 3589. https://doi.org/10.1038/s41467-018-05915-w
Weninger, A., Hatzl, A.-M., Schmid, C., Vogl, T., Glieder, A., 2016. Combinatorial optimization of CRISPR/Cas9 expression enables precision genome engineering in the methylotrophic yeast Pichia pastoris . J. Biotechnol. 235, 139-149. https://doi.org/10.1016/j.jbiotec.2016.03.027
Weninger A, Fischer JE, Raschmanová H, Kniely C, Vogl T, Glieder A. Expanding the CRISPR/Cas9 toolkit for Pichia pastoris with efficient donor integration and alternative resistance markers. J Cell Biochem. 2018 Apr;119(4):3183-3198. doi: 10.1002/jcb.26474. Epub 2017 Dec 26. PMID: 29091307; PMCID: PMC5887973

1. A genetically modified Komagataella phaffii yeast cell for expression of a Protein or Polypeptide of Interest (POI), comprising in its genome a recombinant nucleic acid sequence encoding a POI, and a genetic modification in the open reading frame at any one or more of position 949930 on chromosome 1 (genbank LT962476.1), position 65654 on chromosome 2 (genbank LT962477.1), position 1303485 on chromosome 4 (genbank LT962479.1), position 1323758 on chromosome 1 (genbank LT962476.1) and/or position 1491140 on chromosome 4 (genbank LT962479.1),
wherein said genetic modification is an inactivating modification.

2. The yeast cell of claim 1, wherein the genetic modification is at position 949930 on chromosome 1 (genbank LT962476.1), position 65654 on chromosome 2 (genbank LT962477.1), position 1303485 on chromosome 4 (genbank LT962479.1), position 1323758 on chromosome 1 (genbank LT962476.1) or position 1491140 on chromosome 4 (genbank LT962479.1).

3. The yeast cell of claim 1, wherein the genetic modification is a deletion or insertion of one or more bases, or a fusion of a chromosomal DNA sequence with a sequence of another chromosome.

4. The yeast cell of claim 3, wherein the deletion is a deletion of at least 50%, preferably at least 90%, of the gene at position 949930 on chromosome 1 (genbank LT962476.1), position 65654 on chromosome 2 (genbank LT962477.1) position 1303485 on chromosome 4 (genbank LT962479.1), position 1323758 on chromosome 1 (genbank LT962476.1) or position 1491140 on chromosome 4 (genbank LT962479.1).

5. The yeast cell of claim 3, wherein the insertion is integration of the recombinant nucleic acid sequence encoding the POI.

6. The yeast cell of claim 1, wherein the sequence encoding the POI is comprised in an expression cassette, preferably comprising the following functional regions:
a. a promoter active in yeast of the genus Komagataella,
b. the nucleic acid sequence encoding the POI, operably linked to said promoter,
c. transcription termination sequences, and optionally
d. a selection marker, preferably an antibiotics resistance gene or carbon source utilization marker.

7. A method of producing a recombinant Protein or Polypeptide of Interest (POI) comprising the steps of:
a. providing a genetically modified yeast cell according to claim 1,
b. cultivating said genetically modified yeast cell in a culture medium under conditions that allow for expression of the POI, and
c. isolating the POI from the cells or the culture medium.

8. A genetically modified Komagataella phaffi yeast cell for expression of a variety of Proteins or Polypeptides of Interest (POIs), comprising in its genome
a. a landing pad, comprising an empty expression cassette comprising target sequences for homologous recombination, and optionally any one or more of a selection marker, a stuffer fragment, a promoter 5′ of said stuffer fragment, and a transcription terminator 3′ of said stuffer fragment; and
b. a genetic modification in the open reading frame at position 949930 on chromosome 1 (genbank LT962476.1), position 65654 on chromosome 2 (genbank LT962477.1), position 1303485 on chromosome 4 (genbank LT962479.1), position 1323758 on chromosome 1 (genbank LT962476.1), and/or position 1491140 on chromosome 4 (genbank LT962479.1).

9. The yeast cell of claim 8, wherein the genetic modification is at position 949930 on chromosome 1 (genbank LT962476.1), position 65654 on chromosome 2 (genbank LT962477.1), position 1303485 on chromosome 4 (genbank LT962479.1), position 1323758 on chromosome 1 (genbank LT962476.1) or position 1491140 on chromosome 4 (genbank LT962479.1).

10. The yeast cell of claim 8, wherein the genetic modification is a deletion or an insertion.

11. The yeast cell of claim 10, wherein the deletion is a deletion of at least 50%, preferably at least 90%, of the gene at position 949930 on chromosome 1 (genbank LT962476.1), position 65654 on chromosome 2 (genbank LT962477.1), position 1303485 on chromosome 4 (genbank LT962479.1), position 1323758 on chromosome 1 (genbank LT962476.1) or position 1491140 on chromosome 4 (genbank LT962479.1).

12. The yeast cell of claim 10, wherein the insertion is integration of the landing pad.

13. A method of producing a recombinant Protein or Polypeptide of Interest (POI) comprising the steps of:
a. providing a genetically modified yeast cell according to claim 8,
b. replacing the stuffer fragment with a nucleic acid sequence encoding a POI, preferably using homologous recombination,
c. cultivating said genetically modified yeast cell in a culture medium under conditions that allow for expression of the POI, and
d. isolating the POI from the cells or the culture medium.

14. Use of the yeast cell of claim 1, for producing a recombinant protein or polypeptide of interest (POI).

15. Use of the yeast cell of claim 8, for producing a variety of POIs.

16. Use of the yeast cell of claim 8, for producing a recombinant protein or polypeptide of interest (POI).


SEQUENCE LISTING (24 sequences)

SEQ ID NO: 1 - protein, 261 aa, Artificial Sequence
  OTA57433.1
MKSLSFSLALGFGSTLVYSAPSPSSGWQAPGPNDVRAPCPMLNTLANHGFLPHDGKGITVNKTIDALGSALNIDANLSTLLFGFAATTNPQPNATFFDLDHLSRHNILEHDASLSRQDSYFGPADVFNEAVFNQTKSFWTGDIIDVQMAANARIVRLLTSNLTNPEYSLSDLGSAFSIGESAAYIGILGDKKSATVPKSWVEYLFENERLPYELGFKRPNDPFTTDDLGDLSTQIINAQHFPQSPGKVEKRGDTRCPYGYH

SEQ ID NO: 2 - protein, 265 aa, Artificial Sequence
  XP_001390900.2
MKTTTLLCLAAALTQTYAFPQQGAPHPLPWSPPGPNDVRAPCPMLNTLANHGYLPHNGKDITERHTINALYNALGIEEELAIYLHQEAVTTNPAPNATTFSLNDLSRHDILEHDASLSRQDAYFGDNHDFNQTIFDETRSYWTSPIIDVKQAAVSRQARVNTSMATNPNYTMSELGDSFSYGETAAYIIVLGDKEKGLVNRSRVEYLFENERLPLDLGWSRAKENITFDDLSTMLQRIINATGGESEFDRELAKRGGVHVGSWRG

SEQ ID NO: 3 - protein, 272 aa, Artificial Sequence
  KIK53163.1
MNLLFLSFVTLAISNGVRSDSTTSAQIDWSAHQWKAPGPNDSRGPCPGLNTLANHAFLPRNGSNITIPVVLKAINDGFNFKPINIMRVAAKLGLLTTDAEDNFSLSELALHGTLEHDASLSRNDFALGDNIHFNETVFSTLNNSNPGFDYYDPISAAQVLDQRLAEDKIVNPTLINTIKEFSVRAIESAFYLSVMGNVTTGVAPKNFVQILFREERLPLEEGWKRSEVPIDENTLDNIVNQVQNASNWAASGEVICPWIRLQPEGDVVVVQG

SEQ ID NO: 4 - protein, 273 aa, Artificial Sequence
  XP_020060613.1
MRYFVLACAPLLYAVTLAFPRADYVSEGKLPAGHPPLDWKPAGLGDARAPCPMLNTLANHGYLPHDGKDITKAHTIAALHSALNIDRELAQYLFQEALTTNPAANATTFSLNDLSRHNILEHDASLSRLDYYFGDNHDFNQAIFDQTRQHWPDPIITVQAAANAREARVRTSNATNPTFTLSELGTAFGYGETAAYIIILGNKTSGLVDRSWVEYLFENERLPVELGWTRHEEAVSMDDLEGMMQEVINATGHAEEVKRELVRRGDLHVGRRA

SEQ ID NO: 5 - protein, 262 aa, Artificial Sequence
  OTB17553.1
MKLVYLSSAVAFGSAIADTAPWEGPGPNDVRGPCPMLNTLANHGFLPHDGKNIHVNKTVDALSSALNIDPELGSFLHSFAVTANPQPNATWWNLDHLSRHNILEHDASLSRQDAYFGAPDVFNEAVFNQTKSYWTGDVITLQMAANARLARLMTSNLTNPEYSMSDLGSSFSIGESVAYVAILGSKETRTVPKAYVEYLFEKERLPYELGFKKAETPMTETDLGNLMDELISLQHFPQSPGKIAKRSERPSEKRAEKRCPFH

SEQ ID NO: 6 - protein, 210 aa, Artificial Sequence
  OJJ73116.1
MKTTTLLFLVGALTQTHAFPQQGVPHPLPWSPPGPNDVRAPCPMLNTLANHGFLPHNGKNITQQHTINALYNALGIDAELATYLHQEAVTTNPVPNATTFSLNDLSRHDILEHDASLSRQDAFFGDNHDFNQTIFNQTRSYWTSPIIDVKQAALARQARVNTSMATNPNYTMSELGDAFSYGETAAYIIVLGDKEAGLVNRSRVEYLFEN

SEQ ID NO: 7 - protein, 387 aa, Artificial Sequence
  RXW15716.1
MVSKPFALLTALLLTLGSLSTASAFPNLQAPNGNLLRRQEEASTASQAGGGRPIQAVDPPSPPGPLSFTGTKLVNDRDHPWRPLRNGDIRGPCPGLNTLASHGYLPRDGVATPTQLINAIQEGFNFDHTAAVSATYLGHLLNGNLVTDLLSIGGKTSKTGPPPPPPAHAGGLNVHGTFEGDAGLTRADEFFGDNHSFNQTLFDKLVDFSNRFGGGFYNLTVAGELRYSRIQDSIATNPQFSFKNVRFLTAYGETVFPINLFVDGRQTERKLSLDHAASFFRDMRFPPDFHRAAQPSSAEGVAEVIAAHPWLPGGNEDGQLNNYVVDPNSADFTDPCSLHRFVLGSLQELYPSPTGVLRRNLIKNINFWYTAAFAPAGCPELFPFGQL

SEQ ID NO: 8 - protein, 380 aa, Artificial Sequence
  TFK24496.1
MSLKKLSVLLIALATAADVLAIPQYATLAGLTERQLAEVVPQMNAKIPPAPPGPLRFGGLKLVDDRDHPWRPLRDGDIRGPCPGLNTLASHGYLPRDGVATPTQIINAAQEGLNLINQGAKLATYAALLLEGNVVTNLLSIGGKTHRTGPDPPSPASVGGLSHHGTFEGDASTTRGDAFFGDNHSFNQTLFDQFVDFSNRFGNGYYNYTVGGELRFHRIQDSIATNPEFDLRGFRHSTAFGESAFIANNFVDGRKTGAEAHQLDMDSALSFFRDMRFPRGFHRAAQPGGGEGVDVIFNAHPLQPGHNVGGVNNYVVDTSRGSLFDQCGIYTYMVNTTIRDLYPNPTGVLRRNLNINLDFLYEAFGFTSADCPQVRPFGRN

SEQ ID NO: 9 - protein, 268 aa, Artificial Sequence
  CDP25756.1
MLGIRLVSLLAFTGSALAELDFSKWKTRQPGELRAPCPAMNSLANHGFIQRDGKNITVEGLTPVLKEVFHLSHELAFTVSQLGLFTALDPSKGVFTLQDLTDRHNVFEHDASLSREDAKFGGDQSVLHKGQFQKFMDHFKGEKYISFEAAAKARYAMVQDSRKRNPDFTYDVTHRITSYGETIKYLRTIVEPSTGKCPVDWIKILFEQERLPYNEGWRPPTNELSGFSLASEVLELALITPEKLPVDECLGKGKGKGNCKRRRSYLGI

SEQ ID NO: 10 - protein, 263 aa, Artificial Sequence
  OTB09996.1
MKLTFMSSVVTLGSAVAAYPTSWEAPGPNDVRGPCPMLNTLANHGFLPHDGKNINVNNTAEALSKGLNLAWELGVDLHDFAVMANPQPNATTFDLDHLSRHNVLEHDGSLSRQDAHFGPPDVFNEAVFNQTVSYWTGDVVTMQMAANARLARLMTSNLTNPEYSLSDLGSGFSIGESVVYLLVLGNKDTAEAPKNYLEYWFRNERLPYELGWERPNVIMTGDDLGNAMDKLVTLQHFPQSPGKITSDPEKASAKLAGKRHLFH

SEQ ID NO: 11 - protein, 378 aa, Artificial Sequence
  PPR06026.1
MRLPIFYTAIFSLFTHRVIAFPAHGSLAGLSKEQLEAIIPMLEVREPASPPGPLQDTSAKLVNDKAHPWQPLRPGDIRGPCPGLNTLASHGYLPRNGIASPSQIIEAAQEGFNMLNGLAIFLTYAAHLVDGNMLTDKLSIGGKTPLTGPAPPAPAIVGGLDTHAVFEGDTSMTRADFFFGNNHDFNETLFDEMVKFADKFGAGKYNLTVAGEYRWQRIQDSIATNPTMSFVSPRYFTAYGESVFAINFFVDGRQDDGQLDMKVARGFFQDSRMPDGFFRANGPKGTDGASQVAAAHPIAPGRNVGGVNNYVVDPTSADLSTGCLLYTNFVNQTVRSLYPNPTGALRKALNKNLDFFYSPIIQFEGAASCPQVFPFGKD

SEQ ID NO: 12 - protein, 278 aa, Artificial Sequence
  KAB8223135.1
MKYYLFASAALFVAGTSSFRLPTDGQLSNGRRASIPAGHPPVHWKPAGPGDVRAPCPMLNTLANHGYLPHDGKNITEEHTIRALKLALNIEEELSQYLFQEALTTNPAENATTFSLDDLSRHNILEHDASLSRLDHYFGDNHDFNQEIFDQTRSYWPDPVINVQAAANAREARVKASNATNPTFALSELGLAFGYGETAAYIIILGNKTTGLVDRSWVEYLFENERLPSELGWVRRKDPISFSDLENMLQQVVNATGNSEDTKLDMIKRGDFHTGRRS

SEQ ID NO: 13 - protein, 275 aa, Artificial Sequence
  THV03356.1
MAFKSLLTSLCTAAVLLASVAAQSNNTNSTDLDDHPYIPPGPNDLRGPCPGLNTLANHGYLPRDGRNLNVSVIVDAGFNGYHMEADILGIAAKVATLTSRDPLSFTLDDIKLHGNIEHDASVSRADFITGDNVHFNETIFSVLANANPGVDFYNTTSAGQVQHTRLAQSTATNPNLTNTDKEFFIRSVESAFYLSAMGDPVTGVAPKNFVQIFFREERLPIAEGWKRSEVPINSDTLGPLFNSIAAQSNWTRGEGCHWVMLSPNDPVLRFGAMFQ

SEQ ID NO: 14 - DNA, 1842 bp, Komagataella phaffii
  ALG9
atgttctccaacaggctcttgttgtttggccttttctatctgagactgatatcaaccttctatggtattatttcagactgtgatgaagtgttcaactactgggagccactcaacttcatgcttagagggtttggaaaacagacttgggagtattctccagagtatgccatccgatcttggtcctatctagtgccactttggatagcaggctatccaccattgttcctggatatcccttcttactactttttctactttttcagactactgctggttattttttcattggttgcagaagtcaagttgtaccatagtttgaagaaaaatgtcagcagtaagatcagtttctggtaccttctatttacaaccgttgctccaggaatgtctcatagcacgatagccttattaccatcctcttttgctatggtttgtcacacttttgccattagatacgtcattgattacctacaattaccaacattaatgcgcacaatcagagagactgctgccatctcaccagctcacaaacaacaactagccaactctctcaacaactccagtcaatatttgatagtagcaaaggcagttttttggtacacagtgggtggattactaggatggcctttcgcattagctttggctctgccctttgctatcaccatttttgttcgaaaagtttactacaaggaattgcatcaattggctgttattggcgctctaagcgtgttcatagtccttcttatccttgcatatgtggtccaaattgactcaatatgttacaacaaaacagaattaattcctcttaacattgttctttacaacgtcttgaataccgatgactctgttggtcctgatatttttggcactgagccagtctcatactacatcctcaatttgctgctaaacttcaatttcttagcattgttgggatatgcaagtttgcccttgctgctaatatttaactttctccccgatttcaatttgaggagttccaatgttaaactttttgggcatgagggcggttctaataaacttataactttgtttgccccactatacctttggtctgtcatattttttacccaaccacacaaagaggaaaggtttctttaccccatttatccacttatcactttgggtgctgcatttgctactcatcaattgattcgaattccatcattattatcaactgtatttattccaaacaaaagggttcttcacaaagcaattaatctcacgattatcatcttgttttcatcatttattgttgttatctccgtcttaagagtttttgctcttatttcgcattatagtgctccgttatttgtctatcaacatctggaccagtttgcttcagatactccaaaaaatgtatgtgttggtagagaatggtatcactatccttcttcatttttcctacctccttctatgcgtctacgctttgttaggtcaggctttagtgggatgttaccaggtgattttgacgaatccgtttcaaggctttcttccatgtctaatataccaagagaactgtataataacaaaaatctttttgaatccgacaaggtcatcccctttgaggagtgtgatttttacgttgacatttccaaagctgtggattctgaacaaaaagaaatagcaatattggccccatcctactcagatcaagatcccgcagttttattagatgattggtccttggtacactgtgataagttcatagatcaggatcacagcagaggcctgggaagaatcctttatcttccaaaacgcttccacagaaaaatgaagacaaatctgagataccagcattactgtctattgaaaaagaaaagtagttaa

SEQ ID NO: 15 - DNA, 4617 bp, Komagataella phaffii
  SBR8
atggagaccaacagggctagtgaacactccattcaagttcctcctggagtttacccgctcaacgggcatgactctgtgaacgaaggtcccaaaacagagcttatctacccagatttcaagatttggcagcattctccacaagatgacattataatgactagatatcttcaaaagggtttttttgaagcgcctgtggtccaaaatgagtccttatctgctcgaagtctgatggccgtgagtctccatgaaaactccaacgagcccaatgcatccgaagagcagacagccaaggctccgaatccaaagataaagcttttaagtgattacattatacaagcaattttccaaagacgacagttgagtaagatacacaccaaagtgatagtcagacctcctccaagggtaactctgaccgaacacaagagagaaaaatggatggaaaatttggccaataccagaatccctttgtcccaattagctagagctatcccccatggtatcaagaaccgaatactgctggagcagtgctgtctgtataatgttcctgttgcccgtgctgtctggttggtcaaatgcgtggcaggtaacgaagtcagagtgttgagaaggaaggtccagcagtccagcaccaatgctctttccaccaacgcaattttcaatacctggctttctgattggaccgaccaggttaccaactttttagagcggttcatgattagcatatttactccaaatcaaaaagattggagggaaaggtccaactacaaccttactttggtaggaaatctcttcatggaagatcttttgagacaagacaagttcttgggttggattgtgtcttttttgcgccaatgcttggttaagttatcctctggttcgaaagaagagccagatcatgtatcgcagggattaaacttgcttaccattcatttggtaattctacgtctgttctggttcaaaatcctaaaatcagatcacatatcaaaggaagcaagtgtggtccttttacaattgcatgccaaattagtggacaaaagtacactccagtcgggaaactccccgtcaacatttatcatcgagctcaaggagaacattaattttttgatcaagtatcttttctattacagtcctgatgctttcatcattcccaactattggcatgatttgaaacacacattgaagaaaattttggacacttctaacaacctggttcgaacgcagttcaagcttgtaacctacaggaatgaatccctaattgtcaatctgccgcaatctttaaacaaacccttgaacttgaaccaagatgagcctttgcagagcaacagggatgatctcctcaaatcactggatacgtttttttcctcctctgattctattggcacgttgacgtcgttggtttttgaaaagatagacgaagatgaagaaacagagatcaaaacttttgctccagacatagaacccaatgaaccagaggcctctgagacaatagagatagattcgcctattaagaattctcccattcctaataataaagaagtttcacccccaatcacaaccaagaaaggaaacaagaaggataccagaaagttatggagttacaatttcagactgttgctaaattggtctatatcaaattaccgaaaccaaagctcgtctgttgttcaggtgaatcttgttgtttctattttagaacagaggctacgagaaatttattcaaacacaaccaatcttagagccaaaaagttgaaactggaaattgagcttgagctattggactggatattccagattaacgattcttacaatgttttgaagcagaataacgagacagaaattgtatataaactagagacattggaatatctttgtgcaagatgttaccaatctggattgttgactgtttcatcttacataagaaaacttatcgcttccggagttatctatcttccagattgcttcaaaaatagcacaatccacagcttagttctgaaaagcttacctgttttgaagaataataatttggacaatattctaagaaagctctcaaacaaagaggaaccgtatgatagtgccaaagacatcttattcttcgaagaagcaaaatcaagtttgctcccgtttatcgaatggattgtcagtgattttgagattaaccttgatcctgtctggaactgtttaaagtctctggatactaaaacggctatattagtctctgaatggtttatgagtgagacttttcgtcttctcttcgaacgaactgcaaacaagaaccaagtaaaggttacagagaagaaactaagtttaataacctacgcattggtcgaaaaattaaattgtttacctcagttctacgtgaagctcctgcaggcatttgctcttgagttgaagtcgttttcaatagataaaatgacaatgatatttatctccgagattatccagtttcacgagcaacaggcggctgttttttccgccaaggaagagactaatgcctatacgaatatgatcaagtctttgtttcaggtcgtcgatgagggtaagtatctgatcaactggacagtattcaaaccaacactggtcaatgataaaaatgttatatcagagctttctaagaggattgaactatccaaaaagagttacatcaaggacttatccaacctctatcaacttaacttacgttcatcttcattcttactccataacatgaattccaaagttgagaatttgttgaatttcaacggccttgtacacaacacacacacattagcgggtcgactatgggatatggtcaggaagaaatctttcactaacacagatataccacccgaccagtatgaaagagacctcttccagtcgctgaagctaatgaaattggaaaggccgaatgatttggtcaaagcagttctagcgtatacgaagaaaaacgttttggccacattgtccttggagtacacatcgataactacttatattttgaaactctttttgaatgaagttctagatttggaacaactttgggacttattcagcaccgttaacgatactttcactgacgtcagctgccagaagtttttgaaaggactcatgtttgataagtacttctcagacattgagtgtatccaattgctctcagattttgaacttttgaaattaaagatggttcgttctaaggtgaagcaaacggatttcaagtcatggtgttcaaggatgaggcaatgcttactagaaattgactcaccggaaacaaacaacgggagaacaaattctacccccgtttcaaccgaactggaccaactgcccacctcaaccacaaataatttgcctactccaatgcagttaatgagcactccaactacagtaagcaatttcgatagcatgctctccacgacacctttcaacagcttcttgagtagattcaaagacgaggtcatggagacgtgcaaggagatgatcaatgttcatacggagctttctataaagttttttaacggactaacctcttccttgcaaggggagggtatgagcataagaattctcaatcaacttctgctgcaaaacgatcaagtgatcgtgagttgtaatgatatgtccaaagtactgcaattgctagacccattgaatttgggggtttgccaactattgaccaagaccttacatgaaaaggcttttcagtacaaaagaaaaaagaactctatgaaccacgaagaaaagtccagtttaatggctgatttaacgctatgtttcctgaaagacctcatgaaaatgccgaagtaccaacattctgaccacaaaagtttggagttggtgggagaattgtttagattcctggaacctgatttgaaattgctgctgctgcattcatgcgagctgttttttcttacatccgagatgttcccgttggtaaactccagggaagataatccgggagacaacacactggacatcctttaccttatcatcaacagctgtactagatatgaagacttattttcattatcgtccaagtaccagaacggaattccaattccggacaatcttgttttttcgttgactgtgtcactggagaaaatgatcgggaaatgcgcccggcccaaaagaaagcttgataagcacgccgaacaaaaagcaatcgagtcatccattctcttggtgtgccggatcattctgattcacaagggttacctggtcggattaatactcaaaaggtctacaggtaacatcaataaagactcatttttgcagaatttggtgaaacttttcaattcgaagttcatcaaatccagctctaaactcgagaatttactgtatgacataatcgtttctatcaagattttgattggtgagcacataggctccaccaatcatgagatactgaatgtttcacctccggattacagcaataaacttaattaccttttggagaatgtttccttccaggacggcttgcgtatgagcaatgacagccaagaactattcgttcaaaacacgaagaccaaaaccttgagcaagttctacattcgcccttatgagttgctgcccgagactactacgaagatggaggagagctgtatcaacctgcacctgttcaatgcatccacagaactgaaaaatcctaaatga

SEQ ID NO: 16 - DNA, 2121 bp, Komagataella phaffii
  ACIB2EUKG772803
atgctacaatttattcctgtgctattggcttcactgctcttcaccatagcgcaagctaaatatagtgaccctggtgcaatggctctatacagctgtagttattttagcttcttgtttgattacactcctattacaaaggacacatactgggagataaattgtcattatgaaccagctttccaaactattgcaatatgtttgaatcagtttgacgacggtagctatgctgaaagaggcttccatgctttgcaggaaagttgttcaaagtacggttcactttccaacttgactcaagattactttgaagatcaagccaagaatggatctcagtaccttattcaagcggattcgttcaatgccactgcagacattttgcacaaccctgtccagctcaatctgacatttgttgatctttactacaagtcatatgttcagttttactggaactttgacttttctcaagatacagtggctatcgtgctccatgtttacgttgttggaatactgcttatgtttgtgttggttaacttgatgaaaaaatttggattaattactaagtttaagggaaacagagtgatcaatgcttttagaagatatatatcgcttccggcactagttggaaggaaacatacagaagcagcaggtttctggaaggtcttcactacattgttacccacccgacttgatttttttatcattggaggttatcttgtcgcctacattgtcatgatgagtgttcactacaatgttgcacatgacgtgattttcctcaataagactgaagctagaatcagatattttgctgacagaacaggtattatagcatttgttcaactacctcttttgttcctgtttggaggtcgaaacaacattctctccttctgcactggtctcaacttttccagttttattgtttatcataaatggattgccagaataatggtatttaatgccatcatgcattctgctgcatggacttggttggaaagagactatcttccagaaatgtttgcagacacatattggaagtggggtgtcgttgctaccattgtgtgcagtttgatggttctgttctccttccactgtgttaggaacacaatgtacgagtttttcctctatgcacatataatacttggttgtttgtttgttgtgggcatatggtaccactgcattgaactgggatggatgcaatgggtgtatgcttcaattgccatctgggcatttgacagacttataagattagtaaggattgcttattatggatttccaaaggcaacgatacaggttgttggggaggaaactatgaaggtaagtttcccaaagccatctaggtggaagtctaacccagctaaccatatattcctgtattttggtgataagctaagattcttccaatcccaccccttcacaccagttatctatgatgacaagattgttgtctacattaaagttaagaagggtataactaagaaaatctacgagcaagctgtcaagaccgccaacggacagataacaatgagggttggtgttgagggtccttatgggcattcttctccccttcacaaatatgatagggcccttctggtggctgggggtaacggtataccaggaccgtttgatcatgctttcaagttggttactgacaaaaatcccacaatagctagtcaaaaagtcaagctagtttggatttcccagagctttcacactatcaaatcgttcttacctgagcttgttctgttgaaggaaaaatgtccacaaaataaggctctggaggtgcagatttatttaacgagagaaactcctattccaatgtacgtctctgaagacgattttaaggatactgtaaataaggaaaaagagtcggtatctgatagtttttcactcttggaaattgataatgtctccgttgataacatcaaagattttgtgcactttagttttggaaggcctgacttgaaatcaataattgttgacgaatttactgaagcggaggactgctctgttggtattgtcacttgtgggcctaccaagatggttgacgaaatcagagagttagtagcacataacctaaacaaatgcaagggaagagttgatctgttcgaagaattggagttatggtaa

SEQ ID NO: 17 - DNA, 3324 bp, Komagataella phaffii
  KAP123
atggatcatcagtttcttagtacattgcaggagacactgcagaaaactactattccagattctgcagtcattaaagcggctactaaaagattgcagaccgaattttataccaacccgctagctctaccagctttgatccacactttacaaaatcataatgataagcaaataagacagctagccgctgtagaaggccgcaagttggtacatattcaatggggtaaagttgaagagtctctgaaacaacaggttcgtgactcaatagttgcaagcactttccacgagaaagataaaaacattaggcattcgtcggctcgagtcattgcatctattgcttcgtttgattttgctgaaaacaagtggactgacctacttgctaaactggtttcagtagctaatagcagtgacgaatctcaaaaggttattgcactatttatcttgtattgtatcctggagttaaatattcccgagctagccccaagtacaaaagacttcttaaatttgttccaagacaatattggtcctcacaattcagttgaagttagggttatctcactgctagctcttgactgtattggtctttacttggacgaatctgacagcattgatgcccaggcagccgctcaattcaaatcggtatttccagctatggttgaagtcctgaaatctgtgttagaaattaatgacatagaaaacgccaaaaaggtttttgatgtgattaatgatttcatacttttggacgattccctttttggagataatgtctactatgcaatcgatatggctagagagattgcctccaacatccatattgacgaggaagttagagtttatgctctgtctactcttagctctgtattgtcacacaggaagtccaaagtgtcttcaaagaagcttggtacagatctcaccattatgtgcttgcaaattgcgtccgaagaaatagatgaagaagaggaacttgacaacgaagatctcgaaaatgagaatgaagagagcagcccgaatactgtagctcttagcctgatctccatgttatcccaagagatgcctccgagccaaattattgctccattgttggaagctctgtcacaaggccttgcatctcctaacaagtttgaaagaagaggtgttatcctatcattgggaattgctagtcccggtgctccagactatatcttaacacatttaactaaaatactagaaggaattgtctcttcattgcaggacccggagttgattgttcaggctgctgcattaaaggttttagactcgttaacttcagagcttcatgagcacgtagctgagcactatgagaaactgcttccacttatcatatccattattgactcagccacaaaactttccgtttacaaatatgctaccggtgctttggacagactgatagaatacatggaacatgacgccattgcgaagtatttagaccctttgatgaataaactattccacatgcttgattctgctactacaatttcattgaaatccgcaattgtttcagctattggttctgctgcttttgcctcaggaaagagtttcataccctactttgacagctcaattcgttacttggaaaagtacttatctcaaaatgtcaaagtcgaggagatgtctgctcaagaaattgaactaaaagcattaacgtttgaaaacatttcaacaatggccagggctgtagggtcagaacctttttcaaagtatgcgaagccacttattgatgcttcataccagtcacttagcacacaaagcgagcgtttgagagagtctggattcgcatttatttctaatatggcaaaagtatatggtgctgagttttccgggtttctagatactatagttcctgagatattcaagtcgttgcagcaggaagagtttcagttcaatattgaagaggatgatgagggactaatagacgatgatattgatttggagagcaagtttaatattcacagtggtatcaccattgaaaaagagattgcagctattgcactcgcacaattggccgagggaaccaagaagaactttgccccttacgttaaaccatcgttagagattctcgtcgagcagattgaagccagttatggaatgagagaaactgctgtaagtagtttatggagaattgtcattgcaatgtatcaaaccagcgaccatatggagtatcagataggtgttccagcttcttcctatgtttctgaagaagttctttcaatcatcaagaccgctcgagatgcttcaattactcaattggttaccgaatttgagctgaatatggtgatctccatacttgattgttttagcgaagctttggaaaaagcaggcccagtagtgatcattgataatggtaattcctcagacttggagaagctatgcatagagctaatgaaacttctgaagtcagagcacaactgtcaattagatggtctcgaagagacggctgaagaggaagtagatgggtccgagtctgaaaatcttatatttgattctgctttggaggtcttagtgagcttgtctaaaactttaggtgcagattttttgaagatactctcctccttcaaagataccattatacagaactgttcgtcccagtcccgattccgaaggatcagtgccataggtgctttggctgatatctcttccaatttgaaggcagctaacccttacacggaggagtttttaaacacttttactgaaaaacttcaaaacgacaagtccttagaagttcgtgggaatgcagcttatggtattggttccgtcatcgagagttctgcattagatctttcggacgcctacccccatttgctttctctgttgaaccaactgctggacaaggccgatagaaggtctctgaaaactgaggaaggtgacgatgaaactagggacgttatcagaagagcctttgctaacgcttgcggctgtgtatctcgtatgggtttgaaatcaccggacaatttgcctttggaaaagattctgcccgtcttattggatcatttacctcttactactgcgtttgaggaaaacgtcccaatttttaactttatcatgttcttgtttcaacaaaataatcagctagtacttcgacaaataccaaaaattgttgaagtgtttgaatttgcttttgtcaaggaggttgagcgaaccaagcttatagaagaggcgacacttggtagaaatgaggacattgatagatccaaacagtttcatcaagaagggttgaaagaaaatgtcatcggattcttaaaacaattggaatcttcctatcccagctctgtgtcctcaagcgacgttttgaagaatgtgatttcttga

SEQ ID NO: 18 - DNA, 1230 bp, Komagataella phaffii
  FLO400
atgctaaccaaggttatttcactcgctattttaactgcttcagcctttgctgattctggagagttcactctttggaacttgtcacccggtgacccttacgactcaactttctggggggtatctgaaggtttaatcgtccctgtagaacctggtgtgacttttgttatcactgatgacctacagcttaagactactgatgatcaattcgttactgttggtgaggactccgctctaggtttaggagctgaaggttcggtagaattcagcatcatcaacgaggatggcattacctctctttactacaacggtgagcttgttactgcttacatttgtgagggtgcagaaccccagatttatctcacaggttcagaggaggaccccgaatgtgtttcttacactgtcgctgtgataggcgtagacggcgaagccccaccaactttcccagaggaagacgacgagacaacaacaaccgatgatccaaccgatgagccaaccgatgagccaactgatgagccaactgatgagccaactgatgagccaaccgatgagccaactgatgagccaaccgatgagccaactgatgaaccaactgatgagccaactgatgagccaactgatgagccaaccgatgagccaactgatgagccaaccgatgagccaaccgatgagccaactgaagagccaactgaagagccaactgaagagccaaccgatgaaccaactccacctcctcctcattggggaaatgaaactgtaactgctactaagactgagtatgaaactacaaaagttactatcacttcctgtgaggaaaccaaatgctatgagactacttctgatgcttgggtttctacttgcaccacagaaattggcggaaaggtaactaaaattgttacttggtgcccaatcccatctactccaggtcctaaaccacctaagcctaccaagccaaccgaaactaagccaaccactgttcctgcaccaaccactaagaagccagaaacaccaactactaagaagccagaaacgccagcccctgagaagccagaaaagacaaccactgttattcctccaccaaccactgagaagccatccactttgtctaccagtagtgttactggaagtgttaccattccaactataactgccactggcggtgctggttccaatttcaacttgggtggattaaccgtcggagttgctggtattgcaatggctttgtttgtgtaa

SEQ ID NO: 19 - DNA, 8706 bp, Artificial Sequence
  SRB8
atggagaccaacagggctagtgaacactccattcaagttcctcctggagtttacccgctcaacgggcatgactctgtgaacgaaggtcccaaaacagagcttatctacccagatttcaagatttggcagcattctccacaagatgacattataatgactagatatcttcaaaagggtttttttgaagcgcctgtggtccaaaatgagtccttatctgctcgaagtctgatggccgtgagaaataatgtatctaaacgcaaactccgagctggaaaaatgttaccggcgatgcgcggacaatttagaggcggcgatcaagaaacacctgctgggcgagcagtctggagcacagtcttcgatgggcccgagatcccaccgcgttcctgggtaccgggacgtgaggcagcgcgacatccatcaaatataccaggcgccaaccgagtctctcggaaaacagcttctggatatcttccgctggcggcgcaacgacgaataatagtccctggaggtgacggaatatatatgtgtggagggtaaatctgacagggtgtagcaaaggtaatattttcctaaaacatgcaatcggctgccccgcaacgggaaaaagaatgactttggcactcttcaccagagtggggtgtcccgctcgtgtgtgcaaataggctcccactggtcaccccggattttgcagaaaaacagcaagttccggggtgtctcactggtgtccgccaataagaggagccggcaggcacggagtctacatcaagctgtctccgatacactcgactaccatccgggtctctcagagaggggaatggcactataaataccgcctccttgcgctctctgccttcatcaatcaaatcgaagttcctatactttctagagaataggaacttcagaagagcgaattcggcgcgccggtaagatccaaatcgatgaattgaccaagcactacggtatgaagccagaagactacactgctgtcagatgtggtatgaatgtcgccaagtacatcatcgaagataagattgatgctggtattggtatcgaatgtatgcaacaagtcgaattggaagagtacttggccaagcaaggcagaccagcttctgatgctaaaatgttgagaattgacaagttggcttgcttgggttgctgttgtttctgtaccgttctttacatctgcaacgatgaatttttgaagaaaaaccctgaaaaggtcagaaagttcttgaaagccatcaagaaggcaaccgactacgttctagccgaccctgtgaaggcttggaaagaatacatcgacttcaagcctcaattgaacagctcttcataagcggccgctcaagaggatgtcagaatgccatttgcctgagagatgcaggcttcatttttgatacttttttatttgtaacctatatagtataggattttttttgtcattttgtttcttctcgtacgagcttgctcctgatcagcctatctcgcagcagatgaatatcttgtggtaggggtttgggaaaatcattcgagtttgatgtttttcttggtatttcccactcctcttcagagtacagaagattaagtgagaccttcgtttgtgcgaagcttgcctcgtcccacgcgggtcacccggccagcgacatggaggcccagataccctccttgacagtcttgacgtgcgcagctcacggggcatgatgtgactgtcgcccgtacatttacccatacatccccatgtataatcatttgcatccatacattttgatggccgcgacggcgcaagcaaaaattacggctcctcgctgcagacctgcgagcagggaaacgctcccctcagcaacgcgttgaattctccccacggcgcgcccctgtagagaaatataaaaggttaggatttgcactgaggttcttctttcatatacttccttttaaaatcttgcttaggatacagttctcactcacatccgaacataaacaaaaatgggtaaggaaaagactcacgttaacatgggtaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaataatagcaaggggtaagaaatagagtaactgacaataaaaagattcttgttttcaagaacttgtcatttatagtttttttatattgtagttgttctattttaatcaaatgttagcgtgatttatattttttgcctcgacatcatctgcccagatgcgaagttaagtgcgcagaaagtaatatcatgtcaatcgtatgtgaatgctggtcgctatactgctgtcgattcgatactaacgccgccccagtgtctacctgtcaaatttgccagcgtcaaatgcctccaggatagaatatgctcgaactgttgaagtccatcaacaaggataacccatatgctctatcggcggagaaaacgttcagagccgcttccttccgcagacgtgccccttccactgctagatgagaagtacggggtttagtgtttccaggcctcgtaaatgccgcaataaatgcttccttgggttcgctacgccctcaggcagacgagtttctacaaaacttcaaggaccgccttttcatatatggccaccatcaatatagagccagcgaacgatgatgcattctggtatattgaacgcgagaagttcctatactttctagagaataggaacttccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgtatttctccatgaaaactccaacgagcccaatgcatccgaagagcagacagccaaggctccgaatccaaagataaagcttttaagtgattacattatacaagcaattttccaaagacgacagttgagtaagatacacaccaaagtgatagtcagacctcctccaagggtaactctgaccgaacacaagagagaaaaatggatggaaaatttggccaataccagaatccctttgtcccaattagctagagctatcccccatggtatcaagaaccgaatactgctggagcagtgctgtctgtataatgttcctgttgcccgtgctgtctggttggtcaaatgcgtggcaggtaacgaagtcagagtgttgagaaggaaggtccagcagtccagcaccaatgctctttccaccaacgcaattttcaatacctggctttctgattggaccgaccaggttaccaactttttagagcggttcatgattagcatatttactccaaatcaaaaagattggagggaaaggtccaactacaaccttactttggtaggaaatctcttcatggaagatcttttgagacaagacaagttcttgggttggattgtgtcttttttgcgccaatgcttggttaagttatcctctggttcgaaagaagagccagatcatgtatcgcagggattaaacttgcttaccattcatttggtaattctacgtctgttctggttcaaaatcctaaaatcagatcacatatcaaaggaagcaagtgtggtccttttacaattgcatgccaaattagtggacaaaagtacactccagtcgggaaactccccgtcaacatttatcatcgagctcaaggagaacattaattttttgatcaagtatcttttctattacagtcctgatgctttcatcattcccaactattggcatgatttgaaacacacattgaagaaaattttggacacttctaacaacctggttcgaacgcagttcaagcttgtaacctacaggaatgaatccctaattgtcaatctgccgcaatctttaaacaaacccttgaacttgaaccaagatgagcctttgcagagcaacagggatgatctcctcaaatcactggatacgtttttttcctcctctgattctattggcacgttgacgtcgttggtttttgaaaagatagacgaagatgaagaaacagagatcaaaacttttgctccagacatagaacccaatgaaccagaggcctctgagacaatagagatagattcgcctattaagaattctcccattcctaataataaagaagtttcacccccaatcacaaccaagaaaggaaacaagaaggataccagaaagttatggagttacaatttcagactgttgctaaattggtctatatcaaattaccgaaaccaaagctcgtctgttgttcaggtgaatcttgttgtttctattttagaacagaggctacgagaaatttattcaaacacaaccaatcttagagccaaaaagttgaaactggaaattgagcttgagctattggactggatattccagattaacgattcttacaatgttttgaagcagaataacgagacagaaattgtatataaactagagacattggaatatctttgtgcaagatgttaccaatctggattgttgactgtttcatcttacataagaaaacttatcgcttccggagttatctatcttccagattgcttcaaaaatagcacaatccacagcttagttctgaaaagcttacctgttttgaagaataataatttggacaatattctaagaaagctctcaaacaaagaggaaccgtatgatagtgccaaagacatcttattcttcgaagaagcaaaatcaagtttgctcccgtttatcgaatggattgtcagtgattttgagattaaccttgatcctgtctggaactgtttaaagtctctggatactaaaacggctatattagtctctgaatggtttatgagtgagacttttcgtcttctcttcgaacgaactgcaaacaagaaccaagtaaaggttacagagaagaaactaagtttaataacctacgcattggtcgaaaaattaaattgtttacctcagttctacgtgaagctcctgcaggcatttgctcttgagttgaagtcgttttcaatagataaaatgacaatgatatttatctccgagattatccagtttcacgagcaacaggcggctgttttttccgccaaggaagagactaatgcctatacgaatatgatcaagtctttgtttcaggtcgtcgatgagggtaagtatctgatcaactggacagtattcaaaccaacactggtcaatgataaaaatgttatatcagagctttctaagaggattgaactatccaaaaagagttacatcaaggacttatccaacctctatcaacttaacttacgttcatcttcattcttactccataacatgaattccaaagttgagaatttgttgaatttcaacggccttgtacacaacacacacacattagcgggtcgactatgggatatggtcaggaagaaatctttcactaacacagatataccacccgaccagtatgaaagagacctcttccagtcgctgaagctaatgaaattggaaaggccgaatgatttggtcaaagcagttctagcgtatacgaagaaaaacgttttggccacattgtccttggagtacacatcgataactacttatattttgaaactctttttgaatgaagttctagatttggaacaactttgggacttattcagcaccgttaacgatactttcactgacgtcagctgccagaagtttttgaaaggactcatgtttgataagtacttctcagacattgagtgtatccaattgctctcagattttgaacttttgaaattaaagatggttcgttctaaggtgaagcaaacggatttcaagtcatggtgttcaaggatgaggcaatgcttactagaaattgactcaccggaaacaaacaacgggagaacaaattctacccccgtttcaaccgaactggaccaactgcccacctcaaccacaaataatttgcctactccaatgcagttaatgagcactccaactacagtaagcaatttcgatagcatgctctccacgacacctttcaacagcttcttgagtagattcaaagacgaggtcatggagacgtgcaaggagatgatcaatgttcatacggagctttctataaagttttttaacggactaacctcttccttgcaaggggagggtatgagcataagaattctcaatcaacttctgctgcaaaacgatcaagtgatcgtgagttgtaatgatatgtccaaagtactgcaattgctagacccattgaatttgggggtttgccaactattgaccaagaccttacatgaaaaggcttttcagtacaaaagaaaaaagaactctatgaaccacgaagaaaagtccagtttaatggctgatttaacgctatgtttcctgaaagacctcatgaaaatgccgaagtaccaacattctgaccacaaaagtttggagttggtgggagaattgtttagattcctggaacctgatttgaaattgctgctgctgcattcatgcgagctgttttttcttacatccgagatgttcccgttggtaaactccagggaagataatccgggagacaacacactggacatcctttaccttatcatcaacagctgtactagatatgaagacttattttcattatcgtccaagtaccagaacggaattccaattccggacaatcttgttttttcgttgactgtgtcactggagaaaatgatcgggaaatgcgcccggcccaaaagaaagcttgataagcacgccgaacaaaaagcaatcgagtcatccattctcttggtgtgccggatcattctgattcacaagggttacctggtcggattaatactcaaaaggtctacaggtaacatcaataaagactcatttttgcagaatttggtgaaacttttcaattcgaagttcatcaaatccagctctaaactcgagaatttactgtatgacataatcgtttctatcaagattttgattggtgagcacataggctccaccaatcatgagatactgaatgtttcacctccggattacagcaataaacttaattaccttttggagaatgtttccttccaggacggcttgcgtatgagcaatgacagccaagaactattcgttcaaaacacgaagaccaaaaccttgagcaagttctacattcgcccttatgagttgctgcccgagactactacgaagatggaggagagctgtatcaacctgcacctgttcaatgcatccacagaactgaaaaatcctaaatga

SEQ ID NO: 20 - protein, 1538 aa, Komagataella phaffii
  SRB8
METNRASEHSIQVPPGVYPLNGHDSVNEGPKTELIYPDFKIWQHSPQDDIIMTRYLQKGFFEAPVVQNESLSARSLMAVSLHENSNEPNASEEQTAKAPNPKIKLLSDYIIQAIFQRRQLSKIHTKVIVRPPPRVTLTEHKREKWMENLANTRIPLSQLARAIPHGIKNRILLEQCCLYNVPVARAVWLVKCVAGNEVRVLRRKVQQSSTNALSTNAIFNTWLSDWTDQVTNFLERFMISIFTPNQKDWRERSNYNLTLVGNLFMEDLLRQDKFLGWIVSFLRQCLVKLSSGSKEEPDHVSQGLNLLTIHLVILRLFWFKILKSDHISKEASVVLLQLHAKLVDKSTLQSGNSPSTFIIELKENINFLIKYLFYYSPDAFIIPNYWHDLKHTLKKILDTSNNLVRTQFKLVTYRNESLIVNLPQSLNKPLNLNQDEPLQSNRDDLLKSLDTFFSSSDSIGTLTSLVFEKIDEDEETEIKTFAPDIEPNEPEASETIEIDSPIKNSPIPNNKEVSPPITTKKGNKKDTRKLWSYNFRLLLNWSISNYRNQSSSVVQVNLVVSILEQRLREIYSNTTNLRAKKLKLEIELELLDWIFQINDSYNVLKQNNETEIVYKLETLEYLCARCYQSGLLTVSSYIRKLIASGVIYLPDCFKNSTIHSLVLKSLPVLKNNNLDNILRKLSNKEEPYDSAKDILFFEEAKSSLLPFIEWIVSDFEINLDPVWNCLKSLDTKTAILVSEWFMSETFRLLFERTANKNQVKVTEKKLSLITYALVEKLNCLPQFYVKLLQAFALELKSFSIDKMTMIFISEIIQFHEQQAAVFSAKEETNAYTNMIKSLFQVVDEGKYLINWTVFKPTLVNDKNVISELSKRIELSKKSYIKDLSNLYQLNLRSSSFLLHNMNSKVENLLNFNGLVHNTHTLAGRLWDMVRKKSFTNTDIPPDQYERDLFQSLKLMKLERPNDLVKAVLAYTKKNVLATLSLEYTSITTYILKLFLNEVLDLEQLWDLFSTVNDTFTDVSCQKFLKGLMFDKYFSDIECIQLLSDFELLKLKMVRSKVKQTDFKSWCSRMRQCLLEIDSPETNNGRTNSTPVSTELDQLPTSTTNNLPTPMQLMSTPTTVSNFDSMLSTTPFNSFLSRFKDEVMETCKEMINVHTELSIKFFNGLTSSLQGEGMSIRILNQLLLQNDQVIVSCNDMSKVLQLLDPLNLGVCQLLTKTLHEKAFQYKRKKNSMNHEEKSSLMADLTLCFLKDLMKMPKYQHSDHKSLELVGELFRFLEPDLKLLLLHSCELFFLTSEMFPLVNSREDNPGDNTLDILYLIINSCTRYEDLFSLSSKYQNGIPIPDNLVFSLTVSLEKMIGKCARPKRKLDKHAEQKAIESSILLVCRIILIHKGYLVGLILKRSTGNINKDSFLQNLVKLFNSKFIKSSSKLENLLYDIIVSIKILIGEHIGSTNHEILNVSPPDYSNKLNYLLENVSFQDGLRMSNDSQELFVQNTKTKTLSKFYIRPYELLPETTTKMEESCINLHLFNASTELKNPK

SEQ ID NO: 21 - protein, 613 aa, Komagataella phaffii
  ALG9
MFSNRLLLFGLFYLRLISTFYGIISDCDEVFNYWEPLNFMLRGFGKQTWEYSPEYAIRSWSYLVPLWIAGYPPLFLDIPSYYFFYFFRLLLVIFSLVAEVKLYHSLKKNVSSKISFWYLLFTTVAPGMSHSTIALLPSSFAMVCHTFAIRYVIDYLQLPTLMRTIRETAAISPAHKQQLANSLNNSSQYLIVAKAVFWYTVGGLLGWPFALALALPFAITIFVRKVYYKELHQLAVIGALSVFIVLLILAYVVQIDSICYNKTELIPLNIVLYNVLNTDDSVGPDIFGTEPVSYYILNLLLNFNFLALLGYASLPLLLIFNFLPDFNLRSSNVKLFGHEGGSNKLITLFAPLYLWSVIFFTQPHKEERFLYPIYPLITLGAAFATHQLIRIPSLLSTVFIPNKRVLHKAINLTIIILFSSFIVVISVLRVFALISHYSAPLFVYQHLDQFASDTPKNVCVGREWYHYPSSFFLPPSMRLRFVRSGFSGMLPGDFDESVSRLSSMSNIPRELYNNKNLFESDKVIPFEECDFYVDISKAVDSEQKEIAILAPSYSDQDPAVLLDDWSLVHCDKFIDQDHSRGLGRILYLPKRFHRKMKTNLRYQHYCLLKKKSS

SEQ ID NO: 22 - protein, 706 aa, Komagataella phaffii
  ACIB2EUKG772803
MLQFIPVLLASLLFTIAQAKYSDPGAMALYSCSYFSFLFDYTPITKDTYWEINCHYEPAFQTIAICLNQFDDGSYAERGFHALQESCSKYGSLSNLTQDYFEDQAKNGSQYLIQADSFNATADILHNPVQLNLTFVDLYYKSYVQFYWNFDFSQDTVAIVLHVYVVGILLMFVLVNLMKKFGLITKFKGNRVINAFRRYISLPALVGRKHTEAAGFWKVFTTLLPTRLDFFIIGGYLVAYIVMMSVHYNVAHDVIFLNKTEARIRYFADRTGIIAFVQLPLLFLFGGRNNILSFCTGLNFSSFIVYHKWIARIMVFNAIMHSAAWTWLERDYLPEMFADTYWKWGVVATIVCSLMVLFSFHCVRNTMYEFFLYAHIILGCLFVVGIWYHCIELGWMQWVYASIAIWAFDRLIRLVRIAYYGFPKATIQVVGEETMKVSFPKPSRWKSNPANHIFLYFGDKLRFFQSHPFTPVIYDDKIVVYIKVKKGITKKIYEQAVKTANGQITMRVGVEGPYGHSSPLHKYDRALLVAGGNGIPGPFDHAFKLVTDKNPTIASQKVKLVWISQSFHTIKSFLPELVLLKEKCPQNKALEVQIYLTRETPIPMYVSEDDFKDTVNKEKESVSDSFSLLEIDNVSVDNIKDFVHFSFGRPDLKSIIVDEFTEAEDCSVGIVTCGPTKMVDEIRELVAHNLNKCKGRVDLFEELELW

SEQ ID NO: 23 - protein, 1107 aa, Komagataella phaffii
  KAP123
MDHQFLSTLQETLQKTTIPDSAVIKAATKRLQTEFYTNPLALPALIHTLQNHNDKQIRQLAAVEGRKLVHIQWGKVEESLKQQVRDSIVASTFHEKDKNIRHSSARVIASIASFDFAENKWTDLLAKLVSVANSSDESQKVIALFILYCILELNIPELAPSTKDFLNLFQDNIGPHNSVEVRVISLLALDCIGLYLDESDSIDAQAAAQFKSVFPAMVEVLKSVLEINDIENAKKVFDVINDFILLDDSLFGDNVYYAIDMAREIASNIHIDEEVRVYALSTLSSVLSHRKSKVSSKKLGTDLTIMCLQIASEEIDEEEELDNEDLENENEESSPNTVALSLISMLSQEMPPSQIIAPLLEALSQGLASPNKFERRGVILSLGIASPGAPDYILTHLTKILEGIVSSLQDPELIVQAAALKVLDSLTSELHEHVAEHYEKLLPLIISIIDSATKLSVYKYATGALDRLIEYMEHDAIAKYLDPLMNKLFHMLDSATTISLKSAIVSAIGSAAFASGKSFIPYFDSSIRYLEKYLSQNVKVEEMSAQEIELKALTFENISTMARAVGSEPFSKYAKPLIDASYQSLSTQSERLRESGFAFISNMAKVYGAEFSGFLDTIVPEIFKSLQQEEFQFNIEEDDEGLIDDDIDLESKFNIHSGITIEKEIAAIALAQLAEGTKKNFAPYVKPSLEILVEQIEASYGMRETAVSSLWRIVIAMYQTSDHMEYQIGVPASSYVSEEVLSIIKTARDASITQLVTEFELNMVISILDCFSEALEKAGPVVIIDNGNSSDLEKLCIELMKLLKSEHNCQLDGLEETAEEEVDGSESENLIFDSALEVLVSLSKTLGADFLKILSSFKDTIIQNCSSQSRFRRISAIGALADISSNLKAANPYTEEFLNTFTEKLQNDKSLEVRGNAAYGIGSVIESSALDLSDAYPHLLSLLNQLLDKADRRSLKTEEGDDETRDVIRRAFANACGCVSRMGLKSPDNLPLEKILPVLLDHLPLTTAFEENVPIFNFIMFLFQQNNQLVLRQIPKIVEVFEFAFVKEVERTKLIEEATLGRNEDIDRSKQFHQEGLKENVIGFLKQLESSYPSSVSSSDVLKNVIS

SEQ ID NO: 24 - protein, 409 aa, Komagataella phaffii
  FLO400
MLTKVISLAILTASAFADSGEFTLWNLSPGDPYDSTFWGVSEGLIVPVEPGVTFVITDDLQLKTTDDQFVTVGEDSALGLGAEGSVEFSIINEDGITSLYYNGELVTAYICEGAEPQIYLTGSEEDPECVSYTVAVIGVDGEAPPTFPEEDDETTTTDDPTDEPTDEPTDEPTDEPTDEPTDEPTDEPTDEPTDEPTDEPTDEPTDEPTDEPTDEPTDEPTDEPTEEPTEEPTEEPTDEPTPPPPHWGNETVTATKTEYETTKVTITSCEETKCYETTSDAWVSTCTTEIGGKVTKIVTWCPIPSTPGPKPPKPTKPTETKPTTVPAPTTKKPETPTTKKPETPAPEKPEKTTTVIPPPTTEKPSTLSTSSVTGSVTIPTITATGGAGSNFNLGGLTVGVAGIAMALFV